(19) United States
(12) Patent Application Publication
(10) Pub. No.: US 2013/0172759 A1
(43) Pub. Date: Jul. 4, 2013

(54) Systems And Methods For Using Photoplethysmography In The Administration Of Narcotic Reversal Agents

(71) Applicants: Richard J. Melker, Gainesville, FL (US); Donn M. Dennis, Gainesville, FL (US)

(72) Inventors: Richard J. Melker, Gainesville, FL (US); Donn M. Dennis, Gainesville, FL (US)

(21) Appl. No.: 13/713,666

(22) Filed: Dec. 13, 2012

Related U.S. Application Data
(60) Provisional application No. 61/570,501, filed on Dec. 14, 2011.

(30) Foreign Application Priority Data
Aug. 8, 2011 (US) PCT/US11/46943

(51) Int. Cl. A61B 5/00; A61B 5/1455; A61B 5/02; A61B 5/113; A61B 5/01; A61B 5/0464; A61B 5/145; A61B 5/20; A61B 5/0476; A61B 5/0496; A61B 5/11; A61M 15/00; A61M 16/00; A61M 31/00; A61M 5/168; A61M 16/12; A61M 16/14; A61B 5/0205

(52) U.S. Cl. CPC A61B 5/4839; A61B 5/4848; A61B 5/0002; A61B 5/02055; A61B 5/6819; A61B 5/682; A61B 5/6817; A61B 5/14551; A61B 5/02007; A61B 5/1135; A61B 5/01; A61B 5/0464; A61B 5/4806; A61B 5/14546; A61B 5/14517; A61B 5/20; A61B 5/0476; A61B 5/0496; A61B 5/1106; A61M 15/0065; A61M 16/0057; A61M 31/007; A61M 5/168; A61M 16/12; A61M 16/14; A61B 5/746
USPC 600/476

(57) ABSTRACT

Provided according to embodiments of the present invention are methods of monitoring and treating respiratory depression that include securing a photoplethysmography (PPG) sensor to a central source site of an individual; administering a central nervous system (CNS) depressant to the individual; processing PPG signals front the PPG sensor with a computer in communication with the PPG sensor; and administering a narcotic reversal agent to the individual if the PPG signals or a physiological parameter derived therefrom are outside a preset value range. Related systems are also described.

### CROSS-REFERENCE TO RELATED APPLICATIONS

This application claims the benefit of U.S. Provisional Application No. 61/570,501, filed Dec. 14, 2011, which claims priority to PCT application No. PCT/US11/46943, filed Aug. 8, 2011, the disclosure of each of which is hereby incorporated by reference in its entirety.

### FIELD OF THE INVENTION

The present invention relates to systems and methods for increasing safety in the administration of central nervous system depressants.

### BACKGROUND OF THE INVENTION

Opioids and other analgesic agents are frequently administered to patients to treat acute and chronic pain. While such agents are generally administered without complications, in some cases, the opioids, either alone or in combination with other drugs and/or a patient's underlying condition, can lead to respiratory depression, a potentially life-threatening condition.

A significant number of cases whereby the administration of opioids has lead to respiratory depression involve the use of Patient Controlled Analgesia (PCA) pumps, which are designed to allow patients to self-administer, for example, opioids. However, instances of respiratory depression have also been seen when analgesics or anesthetics are administered by other methods. While there are some control systems in place which limit the total dose of opioid and/or the frequency with which they are delivered, most dosing algorithms do not take into account all of the varying and relevant factors including, but not limited to, patient size and fitness (e.g., weight), pharmacokinetic interactions that can alter opioid concentration in the blood and pharmacodynamic interactions (patient age, underlying medical conditions, including but not limited to undiagnosed obstructive or central sleep apnea, unusual sleep staging, cardio-respiratory disease and kidney or liver disease, and/or active ingredients of medications in other medical classes) that can markedly alter the biological sensitivity to opioids. Pumps can also be misprogrammed, malfunction, and are generally not able to adjust flow in view of the patient's physiological responses to medications.

In some cases, administration of a narcotic reversal agent such as naloxone can counteract the effects of opioids and thus counteract respiratory depression. However, the respiratory depression needs to be detected in time for such a reversal agent to be effective in preventing adverse outcomes. Conventional monitoring for respiratory depression in the hospital setting involves monitoring, for example, end-tidal carbon dioxide (CO₂). End-tidal CO₂ refers to the concentration of carbon dioxide in exhaled respiratory gases. An end-tidal CO₂ monitor operates on the principle that if sufficient carbon dioxide is not being exhaled, sufficient oxygen is similarly not being inhaled. However, end-tidal CO₂ monitoring may be impractical or inadequate to detect respiratory depression in many scenarios. For example, it may be difficult to measure end-tidal CO₂ in ambulatory patients (non-intubated patients). The equipment for monitoring end-tidal CO₂ may also be relatively expensive and cumbersome to use.

Pulse oximetry has also been used to monitor oxygen saturation levels to diagnose respiratory depression. However, patients are frequently placed on supplemental oxygen due to concerns over opioid-induced respiratory depression. Unfortunately, oxygen desaturation is severely blunted by the use of supplemental oxygen and so pulse oximetry alone may not adequately diagnose respiratory depression.

Thus, current methods of monitoring for respiratory depression have limitations that decrease their effectiveness in diagnosing and/or preventing respiratory depression. Accordingly, there is a pressing unmet need by the medical community for new monitoring systems for the administration of analgesic and anesthetic agents.

### SUMMARY OF THE INVENTION

Provided according to embodiments of the present invention are methods of monitoring and treating respirator); depression that include securing a photoplethysmography (PPG) sensor to a central source site of an individual; administering a central nervous system (CNS) depressant to the individual; processing PPG signals from the PPG sensor with a controller in communication with the PPG sensor; and administering a narcotic reversal agent to the individual if the PPG signals or a physiological parameter derived therefrom are outside a preset value range. Physiological parameters include, for example, respiration rate and respiratory effort.

In some embodiments of the invention, the methods further include securing to the individual an additional sensor configured to determine at least one parameter selected from respiration rate, end-tidal carbon dioxide content, blood pressure, heart rate and heart rate variability. In such cases, in some embodiments, the narcotic reversal agent is administered if (a) the PPG signals or a physiological parameter derived therefrom are outside a first preset value range; and (b) a parameter determined by the additional sensor is outside a second preset value range. In some embodiments of the invention, the methods further include measuring a concentration of a component in the individual's breath. In some cases, the component in the individual's breath includes the CNS depressant and/or a metabolite of the CNS depressant.

In further embodiments of the invention, methods include securing to the individual an apparatus configured to supply oxygen, and in some cases, administering oxygen to the individual if the PPG signals or a physiological parameter derived therefrom are outside the preset value range. In some embodiments, methods include directing the device administering the CNS depressant to decrease the supply of the CNS depressant to the individual if the PPG signals or a physiological parameter derived therefrom, are outside the preset value range. Methods also may include impinging a feed line of the CNS depressant-administering device if the PPG signals or a physiological parameter derived therefrom are outside the preset value range. Methods may further include alerting medical personnel and/or the individual if the PPG signals or a physiological parameter derived therefrom are outside the preset value range.

Also provided according to embodiments of the invention are systems for monitoring and treating respiratory depression that include a PPG sensor configured to secure to a central source site of an individual; a device configured to administer a narcotic reversal agent to the individual; and a controller configured (1) to receive and process PPG signals from the PPG sensor, and (2) to direct the device to administer the narcotic reversal agent to the individual if the PPG signals or a physiological parameter derived therefrom are outside a preset value range.

### BRIEF DESCRIPTION OF THE DRAWINGS

FIG. 1 provides a schematic representation of a SPOC array according to an embodiment of the invention.

FIG. 2 is a flow-chart showing the steps of a method implemented according to an embodiment of the invention to monitor a subject's breathing rate, breathing effort or both and interventions automatically implemented on detection of reduced breathing rate, increased breathing effort or both.

FIG. 3a shows an occlusion device according to an embodiment of the invention prior to occluding a section of tubing. FIG. 3b shows the occlusion device of FIG. 3a after the section of tubing is occluded. FIG. 3c shows a cross-sectional view of the occlusion device and tubing of FIG. 3a.

FIG. 4a shows a cross-sectional view of an occlusion device according to an embodiment of the invention and a section of tubing prior to the occlusion of the tubing by the device. FIG. 4b shows a cross-sectional view of the occlusion device and tubing of FIG. 4a after the occlusion device occludes the section of tubing. FIG. 4c shows a horizontal view of the occlusion device and tubing of FIG. 4a.

FIG. 5a shows a cross-sectional view of an occlusion device according to an embodiment of the invention and a section of tubing prior to the occlusion of the tubing by the device. FIG. 5b shows a cross-sectional view of the occlusion device and tubing of FIG. 4a after the occlusion device occludes the section of tubing.

FIG. 6 provides an overall view of one embodiment of the monitoring system that includes a SPOC array and an infusion pump tubing occlusion device.

FIG. 7 provides a schematic representation of an embodiment of the invention for automatically providing ventilation to a subject on detection of physiological parameters being outside a preset value.

FIG. 8 provides photographic depiction of a user interface according to one embodiment of the invention.

FIG. 9 shows synchronization of PPG and PSG data using a generic alignment algorithm according to an embodiment of the invention to optimally match the PPG AC signal with the PSG ECG signal.

FIG. 10 shows the optimization of individual parameters according to an embodiment of the invention: (a) AUC for Nasal Pressure Drop across different types of events; (b) AUC for Saturation Drop across different types of events; (c) AUC for Pleth DC Drop across different types of events; and (d) Clustering capabilities of DC Drop. Notice that DC Drop separates post-events from normals and events.

FIG. 11 shows saturation differences between a PPG probe placed at a Central Source Site (CSS), in this case, a nasal alar site, as compared with a Peripheral Source/Sensing Site (PSS), in this case, a finger, showing, in (a) optimal time shifts between finger and alar saturation, and in (b) ROC curve of event prediction using finger and alar saturations.

FIG. 12 shows correlation between a SPOC model and a scored RDI.

FIG. 13 shows the leave-one-out performance for a model according to an embodiment of the invention: (a) Correlation of predicted versus actual RDI using leave-one out performance, r=0.933; (b) Correlation of predicted versus actual RDI using all 15 patients in training set. r=0.937.

FIG. 14 shows amplitude and variance of weights derived from leave-five-out analysis.

FIG. 15 shows the contribution of each channel to the model's output.

FIG. 16 shows the performance of a pleth-only model: (a) Correlation plot and Bland-Altman plot; (b) ROC curves for RDI>10, 20, 30.

FIG. 17 shows an example of diagnostic agreement in correlation plot.

FIG. 18 shows validation results for a SPOC model: (a) Correlation and Bland-Altman plots for all 15 validation patients; (b) Correlation and Bland-Altman plots for 12 validation patients with RDI<80.

FIG. 19 shows ROC curve for a validation set. All three curves, RDI>10, 15, and 20, are identical.

FIG. 20 shows the performance of ODI model of RDI: (a) Correlation and Bland-Altman plot for the ODI prediction of RDI; (b) AUC for both the ODI and SPOC predictions of RDI>15.

FIG. 21 shows (left panel) the correlation between average respiratory rate as determined by nasal pressure (NAP) and PPG (r2=0.88). The Bland-Altman plot is shown in the right panel.

FIG. 22 shows (left panel) the correlation between respiratory rate as determined by nasal pressure (NAP) and PPG (r²=0.83) in one minute regions across 35 patients. The Bland-Altman plot is shown in the right panel.

FIG. 23 shows (top panel) a histogram of IE ratios calculated from 4,473 one minute regions using nasal pressure. The bottom panel shows a histogram of IE ratios from the same regions using PPG.

FIG. 24 shows a signal with an IE ratio of 1:3 used in the simulation study.

FIG. 25 shows a frequency spectrum of a test breath.

FIG. 26 shows original test signal and the processed respiratory component after the algorithm as been applied.

FIG. 27 shows an embodiment of an assembly to provide positive pressure ventilation and delivery of pharmacologically active agents while acquiring exhaled breath information, as needed, based on signal acquired from a subject.

FIG. 28 shows an embodiment of an assembly to provide positive pressure ventilation and delivery of pharmacologically active agents while acquiring exhaled breath information, as needed, based on signal acquired from a subject.

FIG. 29 shows an embodiment of an assembly to provide positive pressure ventilation and delivery of pharmacologically active agents while acquiring exhaled breath information, as needed, based on signal acquired from a subject.

FIG. 30 provides a schematic representation of an TET ensemble according to an embodiment of the invention.

FIG. 31 provides, for a TET ensemble, an internal schematic representing PD, PK, or PD+PK and other relevant signals from the subject being converted into digital signals, if these are incoming as analog signals, and being processed via a central processing unit utilizing software implementing appropriate algorithms stored in Random Access Memory (RAM) or in Read Only Memory (ROM) or both, and then sending, via integrated or independent signal streams, controller information to the infusion pump.

FIG. 32 provides a schematic representation of a TET ensemble according to an embodiment of the invention.

### DETAILED DISCLOSURE OF SOME EMBODIMENTS OF THE INVENTION

The present invention now will be described more fully hereinafter with reference to the accompanying drawings, in which embodiments of the invention are shown. However, this invention should not be construed as limited to the embodiments set forth herein. Rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

It will be understood that when an element is referred to as being “on” or “adjacent” to another element, it can be directly on or directly adjacent to the other element or intervening elements may also be present. In contrast, when an element is referred to as being “directly on” or “directly adjacent” to another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

It will be understood that, although the terms first, second, etc. may be used herein to describe various elements, these elements should not be limited by these terms. These terms are only used to distinguish one element from another. Thus, a first element discussed below could be termed a second element without departing from the teachings of the present invention.

Embodiments of the present invention are described herein with reference to schematic illustrations of idealized embodiments of the present invention. As such, variations from the shapes of the illustrations as a result, for example, of manufacturing techniques and/or tolerances, are to be expected.

Provided according to embodiments of the present invention are methods and systems for monitoring and treating respiratory depression. These systems and methods use photoplethysmography (PPG) to monitor a patient for signs of respiratory depression in order to determine when to administer a narcotic reversal agent to the patient. Photoplethysmography (PPG) is a deceptively simple method whereby a source of radiation, usually light at a particular wavelength (e.g., a light emitting diode, LED, typically at 940 nm or 660 nm), is coupled with a light detector (e.g., a photo diode, the photodetector) such that light is either detected as it passes through a tissue (transmission PPG) or is reflected from the tissue (reflective PPG). The amount of light that is absorbed (transmission PPG) or scattered/absorbed (reflective PPG) is detected by the photodetector. The photodetector then produces an output waveform that may be used, analyzed and processed, as will be described in further detail below, to provide a number of physiological parameters.

According to some embodiments of the invention, provided are methods of monitoring and treating respiratory depression that include (1) securing a PPG sensor to a central source site of an individual (also referred to herein as the “patient” or “subject”); (2) administering a central nervous system (CNS) depressant to the individual; (3) processing PPG signals from the PPG sensor with a computer in communication with the PPG sensor; and (4) administering a narcotic reversal agent to the individual if the PPG signals or a physiological parameter derived therefrom are outside a preset value.

Any suitable PPG sensor may be used in embodiments described herein. However, in some embodiments, the PPG sensors, systems incorporating such sensors and methods of use of such sensors, which are described in the following references, may be used: U.S. Pat. Nos. 6,909,912, 7,024,235, 7,127,278, 7,785,262 and 7,887,502; U.S. Publication Nos. 2007/0027375, 2008/0058621, 2008/0067132, 2009/0043179 and 2010/0192952; and WO 2004/000114, WO 2012/024401 and WO 2012/024106, the contents of each of which is incorporated herein by reference in its entirety.

The PPG sensor may be applied to any central source site of the individual, and more than one PPG sensor may be applied to the same site and additional PPG sensors may be applied to different central source sites. As used herein, the term “central source site” refers to a site on the body that is above the neck of the individual. Thus, central source sites, include, but are not limited to, the nasal septum (e.g., Kiesselbach's plexus or Little's area), nasal alar, lip, cheek, tongue, pre-auricular, post-auricular, and ear canal. Central source sites may provide a significantly larger signal, and in some cases, a markedly improved signal to noise ratio relative to peripheral sites such as fingers, toes, etc. Such signals may allow for the measurement of a wide range of physiologic parameters that can provide early warning of respiratory and cardiovascular changes. In some embodiments, additional sensors may be applied to peripheral sites as well, as differences in the PPG signal at different sites may provide additional physiological information, as described, for example, in U.S. Pat. No. 6,909,912, incorporated herein by reference in its entirety.

A number of physiological parameters may be obtained from the PPG signals generated by the PPG sensor(s). For example, in some embodiments, the PPG signals are processed to obtain the respiration rate and/or other respiratory parameters such as respiratory effort, inspiration, expiration and the like. Any suitable method may be used to determine these respiratory parameters, but in some embodiments, the methods and systems described in U.S. Pat. No. 7,785,262 (METHOD AND APPARATUS FOR DIAGNOSING RESPIRATORY DISORDERS AND DETERMININIG THE DEGREE OF EXACERBATIONS), hereafter “the '262 patent”, which is incorporated herein by reference in its entirety, are utilized. The '262 patent describes separating out the venous impedance component signal to determine respiratory rate, respiratory effort, inspiration, expiration, and the like. Methods for isolating the venous impedance component signal from the pulsatile arterial signal include the identification of peaks and troughs in plethysmography signals obtained at a central source site of an individual, identifying minima or midpoints between peaks and troughs, and using an interpolated line to represent venous impedance component of the signal. Such methods are also discussed in U.S. Publication No. 2008/0190430, which is also incorporated herein by reference in its entirety.

In particular embodiments, the PPG signals are used to determine the respiratory rate and consistency (e.g., Respiratory Disturbance Indices, RDI's=the number of 10 second pauses per hour, with mild being considered to be 5-15 such events per hour, moderate being 15-30 and severe being anything above 30 per hour). In other particular embodiments, elevation in respiratory effort, hypopnea, central and obstructive apnea, respiratory obstruction index, elevation in blood CO₂, decrease in blood O₂ saturation, increase in expiratory phase of respiration, slowing of the respiratory rate, decrease in movement, increase of respiratory effort indicating airway obstruction, or any other indicator of hypoventilation or hypoxemia, may be measured and monitored. These and other respiratory parameters are described in further detail in Examples 1-3 below.

In some embodiments, the PPG signals may also be used to determine other physiological parameters such as heart rate, arterial and venous oxygen saturation, pulse transit time, pulses wave velocity, endothelial dysfunction, arterial pressure wave shape and amplitude, ankle-brachial index, peripheral artery occlusion, arrhythmias and heart rate variability.

A narcotic reversal agent may be administered to the patient if the PPG signals or a physiological parameter derived therefrom are outside a preset value (also referred to herein as a “preset value range”). In some cases, the narcotic reversal agent is administered to the individual if the PPG signals are outside a preset value range. For example, if the amplitude or frequency of the PPG signals is above or below a preset range, the narcotic reversal agent may be administered, either by a person (e.g., a health care worker) or via an electronic controller (e.g., in a closed loop system). The PPG signals may also be processed such that the pulsatile arterial component is separated from the low frequency components due to venous impedance and respiration. One or more of the separated signals may thus have preset value range in terms of amplitude, frequency of a certain signal component, or other signal parameters.

In some embodiments, the narcotic reversal agent is administered if a physiological parameter derived from the PPG signals is outside a preset value range. If the physiological parameter derived from the PPG signals is outside a preset value range, then PPG signals themselves may also be outside a different preset value range (i.e., abnormal or irregular), and in such cases, the administration of the narcotic reversal agent may be effected based on either or both preset value ranges.

In some embodiments, the respiratory effort of the individual may be outside a preset value range. The respiratory effort may be determined by the PPG signals themselves, or may be determined by the Respiratory Disturbance Index, Respiratory Obstruction Index and the like. In some embodiments, if the respiration rate is less than 8 breaths per minute, it is deemed to be outside the preset value range. Respiration rate and other respiratory parameters may be determined by PPG signals alone or they may also be determined by PPG in combination with information from at least one additional sensor. For example, the respiratory rate and/or effort may be determined by using the PPG sensor in tandem with a nasal pressure or nasal flow indicator. Nasal pressure fluctuations may permit accurate measures of breathing rate to be determined even when breathing via the mouth, and the nasal pressure waveform shape may indicate characteristics of the breathing, such as the gradual increase in occlusion or resistance during exhalation or inhalation. The respiratory effort may be determined by a first preset value range of respiratory parameters as determined by the PPG signals and a second preset value range of respiratory parameters as determined by the nasal pressure or nasal flow sensor.

Analogous to respiration and respiratory effort, preset values with respect to other PPG-derived parameters may be established by determining a range of normal values for the parameter and using that range as a preset value range. A deviation from this preset value may alone, or in combination with other parameters, trigger a person or an electronic controller to administer a narcotic reversal agent.

As described above, in some embodiments of the invention, methods of monitoring and treating respiratory depression include securing to the individual at least one additional sensor. In some embodiments, the narcotic reversal agent is administered if (a) the PPG signals or a physiological parameter derived therefrom are outside a first preset value; and (b) a parameter determined by an additional sensor is outside a second preset value. The additional sensor(s) may be configured to determine the same parameters as the PPG sensor (e.g., respiration rate, etc.) and/or they may be configured to determine parameters that are not derived from the PPG signal. Examples of additional sensors include those that can be used to determine respiration rate, end-tidal carbon dioxide content, blood pressure, heart rate and heart rate variability. Further examples of sensors include accelerometers, nasal pressure (NAP) or flow (NAF) sensors, humidity detectors, temperature detector/thermistors, ECGs, pulse oximeters, capnometers, chest wall and abdominal impedance sensors, polysomnography sensors, drug blood level sensors, nanosensors for breath and sensors for other biological media (e.g., blood, sweat, urine).

Thus, the additional sensors may be used to determine deleterious or adverse cardiac states including, but not limited to, orthostatic hypotension, impaired sympathovagal balance to heart, ventricular tachyarrhythmias such as torsade de pointes, impaired cardiac output such as indicators of congestive heart failure; respiratory states, including, but not limited to impaired ventilation and oxygenation; locomotor activity, including but not limited to sedentary actions, sedation, seizure activity, tremor and general hyperactivity; and key biological indicators of toxicities associated with drug overdosing or normal doses, known as adverse drug reactions (ADRs), which are frequently caused by drug-drug interactions (DDIs) due to pharmacokinetic and/or pharmacodynamic drug interactions.

In particular embodiments, PPG, along with an accelerometer, can be used to monitor the effects of the CNS depressants. An accelerometer may be useful, for example, to determine patient/subject position in order to correct the PPG signal amplitude; determine the degree of locomotion (level of sedentary status) in particular patients, determining whether a patient is making meaningful movements (thus providing a watchdog function, if PPG fails, e.g., sensor falls off during the night or if a patient falls, etc.), determining sleep staging (often referred to as “actigraphy”), determining the presence of seizure activity, assessment of the efficacy of a drug used for a movement disorder such as Parkinson's disease (decrease in tremor); detection of falls or sudden changes in position, and assessing the effect of position on cardiorespiratory parameters (e.g. orthostasis, postural hypotension: common with antihypertensive agents, antipsychotics, Parkinsonism medications).

In particular embodiments, PPG may be used in combination with one or more sensors for conducting polysomnography (PSG). A polysomnogram (PSG) will typically record a minimum of twelve channels requiring a minimum of 22 wire attachments to the patient. In standard PSG, there is a minimum of three channels for the EEG, one or two measure airflow, one or two are for chin muscle tone, one or more for leg movements, two for eye movements (EOG), one or two for heart rate and rhythm, one for oxygen saturation and one each for the belts which measure chest wall movement and upper abdominal wall movement. Respiratory effort is also measured in concert with nasal/oral airflow by the use of belts. These belts expand and contract upon breathing effort. However, this method of respiration may also produce false positives. Some patients will open and close their mouth while obstructive apneas occur. This forces air in and out of the mouth while no air enters the airway and lungs. Thus, the pressure transducer and thermocouple will detect this diminished airflow and the respiratory event may be falsely identified as a hypopnea, or a period of reduced airflow, instead of an obstructive apnea. Snoring may be recorded with a sound probe over the neck, though more commonly the sleep technician will just note snoring as “mild”, “moderate” or “loud” or give a numerical estimate on a scale of 1 to 10. Also, snoring indicates airflow and can be used during hypopneas to determine whether the hypopnea may be an obstructive apnea. Wires for each channel of recorded data lead from the patient and converge into a central box, which in turn is connected to a computer system for recording, storing and displaying the data.

In particular embodiments, PPG may be used in combination with a sensor that can detect and/or determine the concentration of a component in the individual's breath. In some cases, the detected component includes the CNS depressant. In some embodiments, the detected component includes a metabolite of the CNS depressant. Further, in some embodiments, the compound detected in the breath is a marker or taggant that is added to a compound, formulation, or coating or capsule, for example, to the CNS depressant, or an active pharmaceutical compound that is administered to the patient. Any known method of detecting compounds in an individual's breath may be used, but in some cases, breath detection may be effected by the use of the technology described in U.S. Publication Nos. 2004/0081587; 2008/0059226; 2008/0045825; and U.S. Pat. Nos. 7,104,963 and 6,981,947, the contents of each or which is herein incorporated by reference in its entirety.

Thus, the PPG and additional sensors may be used to determine pharmacodynamic (PD) and/or pharmacokinetic (PK) factors. PD parameters involve those relating to how a drug acts on a living organism, including the pharmacologic response and the duration and magnitude of response observed relative to the concentration of the drug at an active site in the organism. PK parameters involve those relating to how a drug is interacting within a body, including but not limited to, mechanisms of drug liberation, absorption, distribution, metabolism, and excretion, onset of action, duration of effect, biotransformation, and effects and routes of excretion of the metabolites of a drug. In other words, PK defines the relationship between drug dose and concentration, whereas PD defines the relationship between drug concentration and biological effects.

It should be noted that by combining measurements of selected PD and/or PK parameters, it may be possible to obtain total “snapshots” of the physical status of the subject at any given time that incorporate external effects (e.g., gravity, low oxygen, high smoke or pollution) and internal parameters (hypovolemia, anemia, any drugs operating in the metabolic pathways of the subject, etc) to determine whether the administration of a narcotic reversal agent and/or additional medical intervention is needed. While the term “snapshot” implies an instantaneous reading, “trends” and detection of changes in trends are also amenable to analysis according to this invention. Trend analysis may be particularly important for PPG signal analysis, since plethysmography data is generally calibrated. Thus, the preset value range may be a particular trend or rate of change, and thus, is not necessarily a particular value.

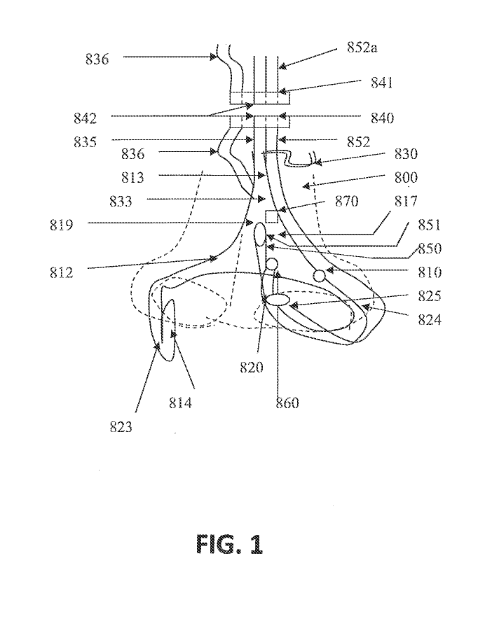

Provided according to particular embodiments of the invention, a PPG sensor and at least one additional sensor may be combined into a single point of contact (SPOC) apparatus (also referred to as a “SPOC array”). A SPOC apparatus according to a particular embodiment of the invention is shown in FIG. 1. In this embodiment, integral with the acquisition of nasal pressure and PPG signals of the subject, the nasal sub-system is also adapted to deliver agents (e.g., the CNS depressant and/or narcotic reversal agent) in fluid, gas, aerosol and/or non-aerosol form to the nasal epithelium. It should be noted, however, that in some cases, the SPOC system may be adapted for emplacement, for example, on the ear of the subject, while the agent delivery subsystem is adapted for delivery to the nasal epithelium. That is to say, the site of the PPG and/or additional sensors and the site of fluid or pharmacologic agent delivery may be the same or different. Where fouling of the sensors by delivery of fluids, gases, aerosols and/or non-aerosols is a likely, it may be desirable to separate the sensors from the site of agent delivery.

Turning to FIG. 1, details are provided for a nasal alar sensor that is integrated with a nasal epithelium agent delivery system. This subsystem is similar to the system 800 described in US2010/0192952, paragraphs 0056-0057, herein incorporated by reference.

A nasal probe embodiment 800 is configured for obtaining plethysmography readings and/or oxygen saturation readings from the user's nasal alar region. The nasal probe embodiment 800 includes a base portion 813 which runs along the longitudinal ridge of the nose. At the distal end 833 of the base portion 813 is a bridge portion 819. The bridge portion 819 runs transversely across the nose and comprises a right flap portion 812 at one end and a left flap portion 817 at its left end. The right and left flap portions 812, 817, respectively, are positioned above the right and left nares of the user. The left flap 817 has attached thereto or integrated therewith at least one LED 810 or other light source. Extending down from the right and left flaps 812, 817 are a right extension 823 and a left extension 824. Attached to or integrated with the left extension 824 is a wing fold 820 that is configured to be inserted into the user's left nostril. The wing fold 820 has at its distal end a photodiode 825 attached thereto or integrated therewith. The wing fold 820 is designed to bend over and be inserted into the user's nostril such that the photodiode 825 is positioned directly across from the LED 810 located on the exterior of the user's' nose. Extension 823 comprises wing fold 814 which is designed to be inserted into the user's right nostril. The positioning of wing fold 814 in the user's right nostril provides a counter force to the wing fold 820 which would tend to pull the probe 800 towards the left. Thus, the right flap 812, right extension 823, and right wing fold 814 act together to assist in securing the nasal probe 800 in place. The nasal probe 800 is provided with an adhesive material 835 and a peel-back layer 830. Before use, the peel-back layer 830 is removed and the adhesive 835 assists in securing the nasal probe 800 to the skin of the user's nose. At the proximal end 842 of the base 813, a connector 840 is provided. Wires 836 are provided in the nasal probe embodiment and run from the LED 810 and photodiode 825 up to connector 840. Furthermore, a flex circuit may be attached to or integrated with the probe embodiment 800 so as to provide the necessary wiring to the LED 810 and photodiode 825.

The connector 840 is adapted to securely mate with connector 841 via clips 842 to thereby provide electrical continuity for wires 836 to wires 836b which connect to the processing elements of the system described elsewhere. Also shown in FIG. 1 is an agent (fluid, aerosol and/or non-aerosol or gas) delivery tube, 850, which runs along the nasal alar assembly into the nose and is oriented toward the intranasal epithelium at its distal end 851. At its proximal end 852, the agent delivery tube 850 is integrated with connector 840 which, when coupled with connector 841, again via clips 842, to sealingly connect with extension 852a which runs to the agent reservoir(s) of the system described elsewhere, and which, on receiving instructions from the controller, also described elsewhere, results in administration to the subject of selected fluids and/or pharmacologically active agents. Of course, more than one separate tube line 850 may be provided, permitting more than one agent or more than one agent combination to be delivered to the subject at any given time. Ideally, the agent delivery tube internal diameter is sufficiently small to minimize any dead space volume while at the same time being sufficiently large to permit ready delivery of agent to the subject.

Another element shown in FIG. 1 is a nasal pressure sensor 860. The nasal pressure sensor detects small changes in pressure near the nasal opening caused by breathing. Typically these changes are quite small (e.g., less than 2-3 cm H₂0; 0.03 PSI) and so the sensor must be very sensitive and accurate. Even during mouth breathing, pressure fluctuations can be detected near the nasal opening, although the pressure changes are even less than described above. Typically, a nasal pressure measurement system includes a small bore sensing line inserted into the nasal opening that connects to a very low pressure sensor located a small distance from the sampling point to minimize pressure losses in the sampling line (although a pressure sensor could be embedded in the nasal opening). Pressure fluctuations measured by the pressure sensor (various types of pressure sensors are common and known to those skilled in the art) are typically temperature compensated and digitized for processing by a digital processing system. In addition to a pressure sensor, flow sensors can also be used. Pressure sensors are typically considered to have more information related to wave shape, but flow sensors can be very simple thermistors or other devices that can be directly inserted into the nasal opening to reduce the need for tubing.

Also shown in FIG. 1 is an ECG lead 860, which provides the system of this invention the ability to secure direct cardiac signals. Along with a second lead which can be attached to the undergarments of the subject or directly to the skin as a conventional ECG electrode is attached, a single lead ECG may be present in the SPOC array. The ECG signal allows not only the detection of the heart rate but also detection of arrhythmias. Several derived signals such as pulse transit time can also be determined by using the ECG signal in conjunction with the PPG signal.

The nasal probe 800 may be dimensioned so that placement onto the fibro-areolar region is optimized for the user. Other features are contemplated as well, including clips, hooks, and reflectance designs for either inside or outside nose, which could be inconspicuous and would be especially advantageous for ambulatory and long term use.

The SPOC array design described above may facilitate closed-loop as well as open-loop delivery of fluids and pharmacologically active agents, non-invasively, to a site of excellent access and bioavailability (the nasal epithelium). It also may allow for improved accuracy for measurements of the subject's breathing patterns (via the nasal pressure transducer sensor) and ECG readings. Of course, in various embodiments, not all of these elements are required to be present. For example, the agent delivery tube and the nasal pressure sensor may be present, while the ECG sensor may be absent or located elsewhere. Likewise, as mentioned above, the agent delivery system may deliver agents to the nasal epithelium, while the SPOC array may be emplaced at the subject's cheek or ear. Alternatively, the SPOC array may be emplaced at the subject's nose, while the agent delivery system delivers agent to the subject at any other convenient site. Those skilled in the art will appreciate that the present system accommodates a large number of permutations and combinations, without departing from the central teachings of this invention. It will also be appreciated that a similar arrangement of components may be included for both nares of a subject as described above, such that there is redundancy in the system and, in addition, there are additional options available for providing different drug combinations to the left and right nasal epithelia.

In the methods described herein, any suitable CNS depressant may be administered to the individual, provided that there is a corresponding narcotic reversal agent that can be administered to counteract, at least in part, the effects of the CNS depressant on the individual's respiratory system. Examples of CNS depressants include tramadol, benzodiazepines such as diazepam, alprazolam, lorazepam, flurazepam; barbiturates such as secobarbital, pentobarbital and Phenobarbital; and opiods such as codeine, oxycodone, fentanyl, alfentanyl, morphine, sufentanil, diamorphine, methadone, levorphanol, pentazocine, propoxyphene, butorphanol, oxymophone, remifentanil, nalbuphine and buprenorphine. The term CNS depressant also includes anesthetic agents. Combinations of different CNS depressants may also be used. In some cases, any medical therapy that depresses cardiorespiratory function depresses in vivo, particularly those centers in the brain (e.g., brainstem) that regulate the respiratory and cardiovascular systems, may be used.

In particular embodiments, the CNS depressant is an opioid. The effect of opioids on cardiorespiratory function have been studied and modeled. Opioids induce cardiorespiratory changes by acting on the brainstem (and to a more limited extent on the cerebral cortex). In humans, opioids may cause respiration to slow and become irregular, which in turn can lead to hypercapnia and hypoxia. Modeling has successfully explained pharmacodynamic and pharmacokinetic interactions between CO₂ and opioids on breathing. With a gradual increase in opioid levels, for example, with a constant rate infusion, progressive respiratory depression causes gradual hypercapnia that contributes to the maintenance of respiration. On the other hand, a fast rise in opioid receptor occupancy resulting from an IV bolus may lead to apnea until the Pa_{CO2} rises to its steady-state value. This explains why drugs with slower receptor binding (e.g., morphine) may be safer than those that bind more quickly (e.g., alfentanil and remifentanil), despite equianalgesic effects. Opioids also depress the HRV and HCVR through depression of central and peripheral chemoreception, as described above. The degree of respiratory depression appears to vary between drugs, even at equianalgesic levels, but there are currently no opioids available that are devoid of respiratory side effects.

Any suitable narcotic reversal agent may be used in the methods and systems described herein. As used herein, the term “narcotic reversal agent” includes any agent that can counteract, at least in part, the effects of a CNS on the individual's respiratory system. Examples of narcotic reversal agents include, for example, naloxone (e.g., Narcan®, Nalone® or Narcanti®), nalmefene (e.g., Revex®), nalbuphine and flumazenil. Combinations of different narcotic reversal agents may also be used, either via a “cocktail” or via separate administration.

The skilled artisan will generally be able to determine the appropriate concentration of the narcotic reversal agent, and the appropriate concentration may be dependent on the size of the individual, the amount of CNS depressant administered, the severity or type of the respiratory distress, etc. As an example, Naloxone® is an opiate antagonist that competitively binds to the opioid receptors. In some embodiments, if the patient is apneic, the patient may be administered 0.4 mg or 1 ampule of naloxone by IV or IM with careful monitoring. If the patient is not apneic but has a falling O₂ stat (rising PaCO₂ in intubated patients) or PPG indications of respiratory depression, the Narcan® or the Naloxone® may be titrated into effect. Naloxone has a relatively short half life (˜20 minutes) so administration may need to be closely monitored. For narcotic reversal using Narcan®, in some embodiments, the dose administered is 1-10 mcg/kg IV push (in some cases, 1/10th of dose recommended for full reversal of narcotic poisoning), and administration may be repeated. For benzodiazepine reversal, flumazenil may be administered, for example, at a dose of 0.01-0.02 mg/kg, and administration may be repeated.

The CNS depressant and the narcotic reversal agent (which may collectively referred to herein as “the medications”) may be administered by any suitable route, including, for example, intravascularly (intravenous or intraarterial), endotracheally, intramuscularly, intraperitoneally, enterally, epidurally, buccally, intraosseously, (e.g., iontophoretic or non-iontophoretic-based), orally, rectally, intravaginally, sublingually, subcutaneous, transdermally, transoccularly, nasally, intraoticly, pulmonary or intrapulmonary (transtracheal, or via metered dose inhalers [MDIs]), intrathecally, neuraxially (central nerves, peripheral nerves), and intracerebrally. The medications may also be administered at two or more different sites.

In particular embodiments, because of the high rate of bioavailability, absorption and low time for effect, delivery to the nasal epithelium is utilized. For example, the medications may be administered to the mucosa of the nasal septum, particularly at Kiesselbach's plexus (also known as “Little's area”), nasal mucosa of the turbinates and the upper posterior nasal septum. This area may have a high rate of bioavailability and absorption and so medication absorbed at this site may act quickly on the individual. The medications may also be in any suitable form, for example, a fluid, a mist, an aerosol, a solid, and the like (including pressurized gases), and may be present with other compounds, such as permeability enhancing compounds.

In some embodiments, the medications are administered intravenously via an infusion pump. Any suitable type of infusion pump may be used, but in some case, the infusion pump is a continuous, intermittent or patient controlled analgesia (PCA) pump. The use of such pumps has lead to a significant number of occurrences of CNS depressant-induced respiratory depression. Reasons for such occurrence include operator errors, patient errors and equipment errors. Operator errors include programming errors, accidental bolus administration during syringe change, inappropriate dose prescription or lockout interval, drug errors (wrong drug or wrong concentration), inappropriate drug selection (i.e., morphine or meperidine in a patient with renal failure) and disconnection or absence of Y-connector (allowing for accumulation of opioid in the IV tubing followed by intermittent bolus delivery). Common patient errors include activation of the PCA pump by others (e.g., family members) and failure to understand the device. Equipment errors include siphoning of drug (pump placed above patient without flow restriction valve or cracking of a glass syringe) and equipment failure resulting in spontaneous activation of drug delivery.

The medications may be administered in an open loop or closed loop modality. In situations where a medication is being delivered to a subject via an infusion pump using a closed-loop system, if the PPG signals and/or physiological parameters derived therefrom, optionally in view of physiological signals or parameters obtained from at least one additional sensor, are outside a preset value range, the narcotic reversal agent may be administered automatically without the need for any external input or authorization, optionally along with other actions described herein (e.g., ventilation, occluding of feed line, etc.). In an open loop system, if the PPG signals and/or physiological parameters derived therefrom, optionally in view of physiological signals or parameters obtained from at least one additional sensor, are outside a preset value range, a health care worker (or other individual) may be alerted, and the dispensation of the narcotic reversal agent would be administered, or its administration would be authorized, by the individual. Thus, in some methods, one or more devices can process the PPG signals and administer a narcotic reversal agent, and in some cases, increase or decrease the administration of the CNS depressant, without external user input, while in other methods, the administration of the narcotic reversal agent may be effected or authorized by a health care worker.

In general, for most IV drugs, it appears that the variability between dose and pharmacological effect is approximately due to equal contributions from variabilities in PK and PD. However, this contribution can vary by drug. In general for controlling IV drug infusions, irrespective of PK versus PD contributions to variabilities in dose-response, it may be preferable to guide drug dosing based on the biological effects of the drug, because it takes into account the multitude of factors that can alter PK and/or PD, and integrates them at the level of biological responsiveness, which in turn controls drug infusion rates, either in a closed loop (machine outputs automatically modifies drug infusion rates) or open loop (human takes system output and modifies drug infusion rate) configuration.

In addition to the administration of a narcotic reversal agent, other actions may be effected if PPG signals and/or a parameter derived therefrom (and optionally those from additional sensor(s)) are outside a preset value range. For example, in such cases, an alert to the individual or medical personnel may be given; oxygen may be supplied or, if oxygen is already being supplied, the oxygen rate may be increased; the pump may be directed to slow or stop delivery of a CNS depressant and/or an occluding device that slows or stops deliver of a CNS depressant may be actuated.

More particularly, in some embodiments of the invention, before, concurrent with, and/or after administration of the narcotic reversal agent, the patient and/or medical personnel may be alerted. For example, in some cases, an alarm may sound when PPG signals or parameters derived therefrom (and optionally those from additional sensor(s)) are outside preset value ranges. This range may be the same or different than the preset value range for dispensation of the narcotic reversal agent. The alarm may be, for example, auditory, visual and/or tactile. In particular embodiments, an alerting device may provide a wisp of air or electrical stimulation to a cheek (e.g., the suborbital and superior malar region of the face) of the individual if, for example, respiration slows or is obstructed, in order to rouse the patient. Auditory clicks or other quieter sounds, or louder more urgent auditory alarms, may be also be used to rouse the individual. Alerts may also be given to a health care worker and/or the alerts to the individual may be monitored by a health care worker.

In some cases, oxygen may be supplied to the individual prior to, concurrent with, and/or after administration of the CNS depressant. In other cases, the patient may not receive supplemental oxygen in connection with the administration of the CNS depressant. In some embodiments, if the PPG signals and/or a physiological parameter derived therefrom are outside a preset value range, the supply of oxygen to the individual may be increased or initiated. For example, if the respiration rate is undesirable low, of if there are respiratory disturbances, the oxygen supply to the individual may be increased or initiated.

For example, in a clinical setting, such as in an Intensive Care Unit (ICU), where the patient is already or could be intubated, ventilation could be modified accordingly, while still deriving the benefit of the additional information available from implementation of the present system. Devices for supplying oxygen (also referred to as “applying positive pressure” or “increasing ventilation”) include, for example, CPAP, BiPAP (Bilevel Positive Airway Pressure) and adaptive servo-ventilation. Such devices may also be configured to monitor end tidal carbon dioxide.

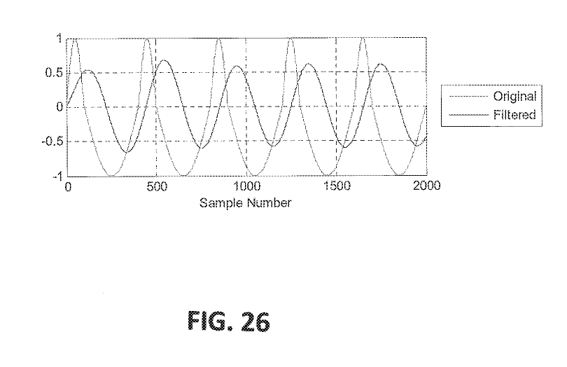

In some embodiments, the device or apparatus for supplying oxygen to the patient includes one or more “nasal pillows,” which are commonly used with home continuous positive airway pressure (CPAP) devices. In some embodiments, the oxygen is supplied by a like means (e.g., tightly sealed masks and similar devices) such as those used to administer CPAP and other forms of “noninvasive positive pressure ventilation.” For hospital applications, the nasal pillows are typically built into a lightweight frame, similar to athletic glasses or the like, with an adjustable band for retaining the pillows in place by placing the band around the rear of the head of the subject (as shown in FIG. 26, described below), with an adjustable fastening means at the back or at another appropriate location, to keep the frame properly positioned on the subject. Materials including, but not limited to, cotton, wool, silicone, latex, foam, and the like, may form the nasal insert portion of the “nasal pillows”, in a fashion analogous to what is commonly utilized for in-ear headphones.

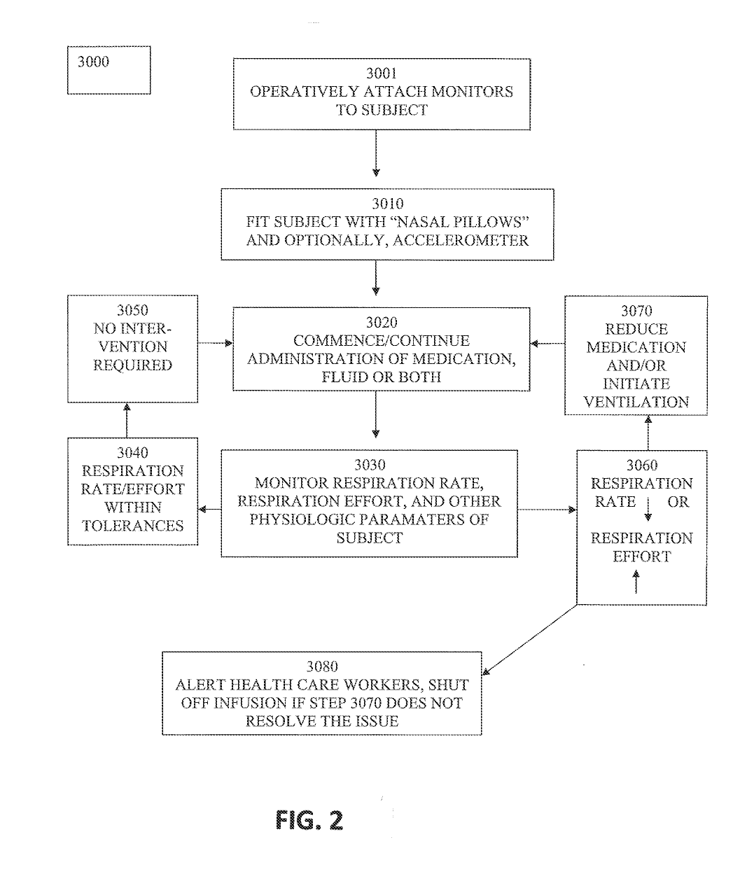

FIG. 2 provides a flow-chart showing the steps of a method implemented according to the system or apparatus of the invention to monitor a subject's breathing rate, breathing effort or both (plus other parameters such as oxygen saturation, end tidal carbon dioxide, heart rate, etc.), and interventions, including administration of oxygen, that may be automatically implemented, for example, on detection of reduced breathing rate, increased breathing effort or both.

In FIG. 2, it can be seen that appropriate monitors/sensors are first attached to a subject at 3001. At a minimum, the appropriate monitors include affixation of a Central Source/Sensing Site (CSS) PPG monitor, placed, for example, on the subject at the nasal alar region. In addition, in some embodiments, additional monitor(s)/sensors may be included.

Once the monitors, including the PPG monitor, are operatively in place on the subject, in a particular embodiment, the subject is also fitted with “nasal pillows”, and optionally, an accelerometer or like device which can record movements of the subject 3010. At this point, administration of medication, fluid or both can be initiated or continued 3020. The subject's respiration rate, effort and other physiologic parameters are monitored 3030, and so long as these parameters remain within pre-programmed tolerances 3040 (preset values) the medical procedure and infusion is permitted to proceed without intervention 3050. However, on detection of a respiration rate drop or a breathing effort increase, or other adverse indicia of subject physiologic condition, 3060, a narcotic reversal agent may be administered and positive pressure ventilation may immediately be initiated and, if necessary, the delivery of medication can be reduced or terminated 3070. Once the adverse condition is resolved, medication/fluid infusion may be continued 3020, and ventilation can be continued or terminated as indicated by the respiration rate signals derived from the CSS PPG monitoring.

In addition to providing oxygen, other medical interventions may be provided, including using cardiovascular assist devices (e.g., automated chest compressors, manual cardiopulmonary resuscitation, intraortic balloon pumps) and administering fluids, including volume expanders and nutrients, e.g., glucose, given via the intravascular route, including intravenously, intraarterially and intraosseously.

Provided in some embodiments of the invention, if the PPG signals and/or physiological parameters derived therefrom (and optionally those from additional sensor(s)) are outside a preset value range, the tubing (or other conduit) between an infusion device and the patient may be partially or completely occluded in order to slow or stop the flow of the CNS depressant. Any suitable occluding devices may be used in combination with the systems and methods described herein. However, in some embodiments, the occluding device is a small device (pneumatic, mechanical or otherwise actuated) that is connected to tubing running between an infusion pump and a patient (also referred to herein as a “feed line”), and which when directed by the controller or individual, acts to “pinch” or “impinge” the tubing, thus disrupting the flow of the opioid (or other medication or fluids) to the patient. Such an occlusion may be temporary, such as until a health care worker intervenes, or may be used more generally to control the flow of the drug. The occluding device may also have its own alarm to alert the patient and/or healthcare workers. In addition, infusion pumps generally include an occlusion sensor which sounds an alarm and shuts of the pump. The occluding device will thus activate the pump's own occlusion sensor and alarm by creating an occlusion.

In some embodiments, the occluding device may be used without requiring any other (e.g., electronic) integration with the fluid/medication delivery system, and can generally be applied to any tubing. As such, the occluding device is an “infusion pump agnostic” solution that does not require imposing design and regulatory burdens on infusion pump manufacturers. It could be a stand-alone monitor for any existing infusion pump system, or it could be incorporated into a third party's next-generation infusion pumps. Thus, while numerous means are known in the art for shutting off flow through infusion tubing, it will be appreciated by those skilled in the art upon reading this patent disclosure that it may be preferable to have a device that shuts off flow by occluding the tubing compared to an in-line solution as there is virtually no chance of contaminating the system with an external shut off mechanism.

Any suitable fluid line occlusion device, when integrated with appropriate physiological monitors according to the present disclosure, may be used with the present invention. Thus, for example, utilizing the physiological monitors described herein, the fluid constriction systems disclosed in U.S. Pat. No. 6,165,151 to Weiner and U.S. Publication No. 2005/0027237 may be adapted for use to the present purposes, and those disclosures are herein incorporated by reference in their entirety for this purpose. Likewise, for example, described in U.S. Pat. No. 6,558,347 to Shuboo et al., incorporated herein by reference, are control devices that permit an infusion tube to be blocked downstream of a pump, and such devices may likewise be adapted for inclusion in the present system, while at the same time relieving the pump manufacturers of the required adaptations of their infusion devices that would otherwise be required to utilize the Shuboo system. Furthermore, and also incorporated by reference for this purpose, there is disclosed by Mabry et al., in U.S. Pat. No. 7,661,440, devices that may likewise be adapted for inclusion in the present system, again without the need for integration to/with an existing fluid infusion system. Other fluid flow restrictors known in the art may also be utilized for this purpose when appropriately adapted for inclusion in the system of the present invention.

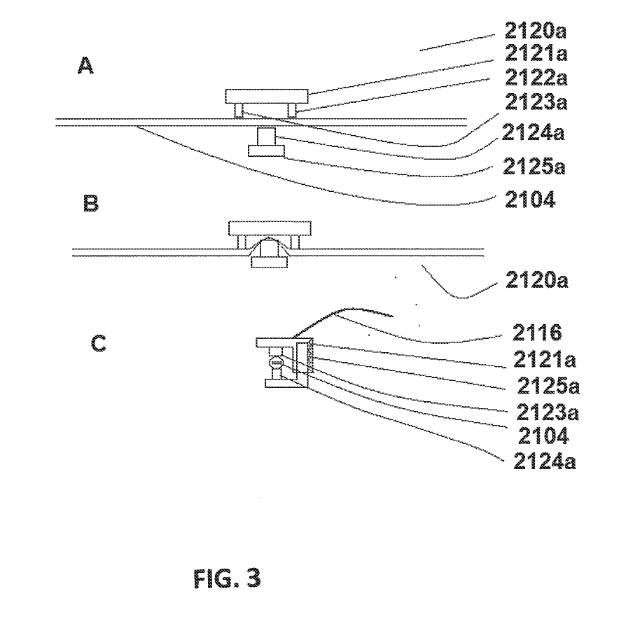

FIGS. 3-5 provide a series of alternate exemplary occlusion devices 2120 for use according to embodiments of this invention. In FIG. 3, there is provided an occlusion device 2120a that includes an upper occlusion member 2121a and a lower occlusion member 2125a. The upper occlusion member 2121a includes two tubing 2104 impingement members, 2122a and 2123a, and the lower occlusion member 2125a includes a single tubing 2104 impingement member 2124a. In FIG. 3A, the occlusion device 2120a is shown in an open configuration, with the tubing 2104 running unimpeded between the occlusion members 2122a, 2123a and 2125a. In FIG. 3B, the same arrangement is shown with occlusion member 2125a impinging from below and occlusion members 2122a and 2123a impinging from above, thereby occluding the tubing 2104 as between these occlusion members. In FIG. 3C, there is shown a side view down the long axis of the tubing 2104, in the occluded state shown in FIG. 3B, with lumen of the tubing 2104 shown as being almost entirely occluded (i.e., the inner lumen of the tubing 2104 is not shown as a circular lumen but rather as a flattened lumen through which very little fluid may pass). FIG. 3C also shows the line 2116 through which the signal has been sent to occlusion device 2120a to actuate the impingement members 2122a, 2123a, and 2124a to be drawn close enough together to either completely or almost completely occlude the lumen of tubing 2104. Those skilled in the art are well aware of many different mechanical and/or pneumatic means for bringing these occlusion members together and to release these members from having been brought into sufficient proximity to each other to thereby occlude the tubing 2104.

As an example, in FIG. 3C, it is shown that the rear element of upper occlusion member 2121a and the rear element of lower occlusion member 2125a are so arranged that the rear element of lower occlusion member 2125a rides within the rear element of upper occlusion member 2121a, and these elements are shown with intermeshed teeth, so that upon actuation, lower occlusion member 125a is drawn upward by intermeshment of the teeth on the rear of its member with the teeth provided for this purpose on the rear of upper occlusion member 2121a. Of course, these two members may be actuated to spread apart, thereby opening the lumen of tubing 2104 to once again permit fluid to flow (or to increase flow) through the tube from the pump to the subject.

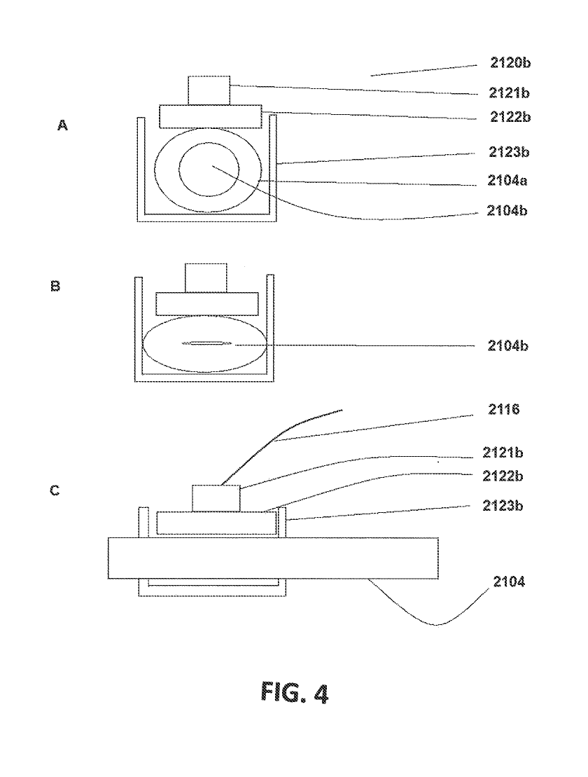

FIG. 4 provides another occluding device 2120 according to an embodiment of the invention. FIG. 4A shows a view down the long axis of the tubing 2104, housed inside an occlusion device 2120b according to this invention. Occlusion device 2120b comprises an upper occlusion member 2121b which is part of a pneumatic system (not shown, but such systems are well known in the art), whereby an upper impingement member 2122b is brought downward to impinge upon tubing 2104 which sits below the upper impingement member 2122b and is held in place by a lower containment vessel 2123b. In this figure, the lumen 2104b of the tubing 2104 can be seen to be wide open, thereby allowing fluid to pass through the lumen 2104b unimpeded.

In FIG. 4B, it can be seen that the upper impingement member 2122b has been pneumatically driven down upon the tubing 2104, thereby occluding the inner lumen 2104b to such an extent that little or no fluid may pass therethrough.

FIG. 4C shows a side view of the tubing 2104 which runs through occlusion device 2120b, such that when the upper occlusion member 2121b is actuated via an appropriate signal transmitted via communication channel 2116, the upper impingement member 2122b may be driven pneumatically to impinge upon the tubing 2104. In so doing, upper impingement member 2122b rides downward within containment chamber 2123b thereby squeezing the tubing 2104 and occluding its inner lumen 2104b as shown in FIG. 4B.

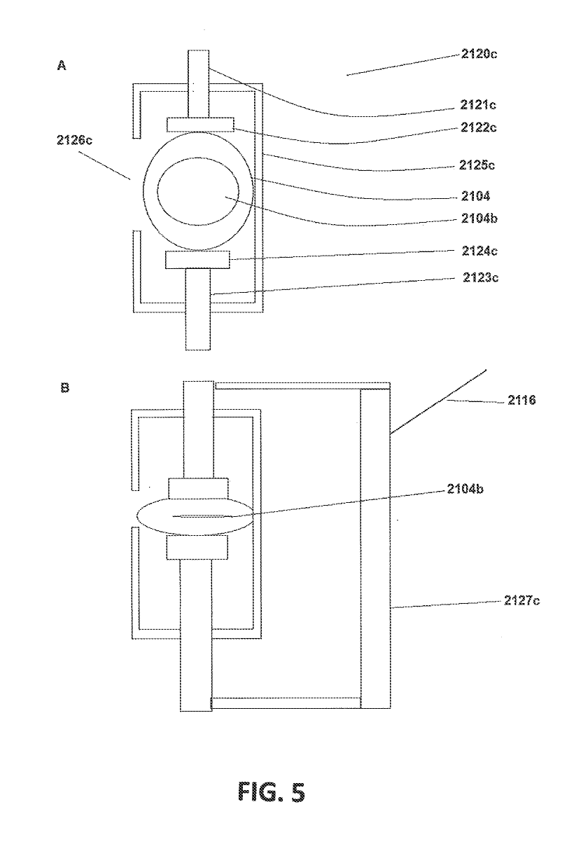

In FIG. 5, in a further exemplary embodiment of the occlusion device 2120, there is provided an occlusion device 2120c that includes upper and lower piston members 2121c and 2123c, respectively, each of which terminates with an impingement member 2122c and 2124c, respectively, which make contact with tubing 2104 arranged there between. The tubing 2104, as well as upper and lower piston members 2121c and 2123c are all housed in housing 2125c, which keeps the tubing 2104 in place and aligns pistons 2121c and 2123c. An opening 2126c is provided in the housing 2125c to facilitate introduction and removal of the tubing 2104 from the housing 2125c. In FIG. 5A, the tubing is shown un-occluded, while in FIG. 5B, the pistons 2121c and 2123c which are integral to a larger pneumatic actuation assembly 2127c, are shown in a position such that the tubing 2104 is occluded, such that its lumen 2104b is so narrow that essentially no fluid whatsoever may pass therethrough. As with the other embodiments of the occlusion device shown in FIGS. 3 and 4, the signal for actuation of the pistons 2121c and 2123c is transmitted via communication channel 2116.

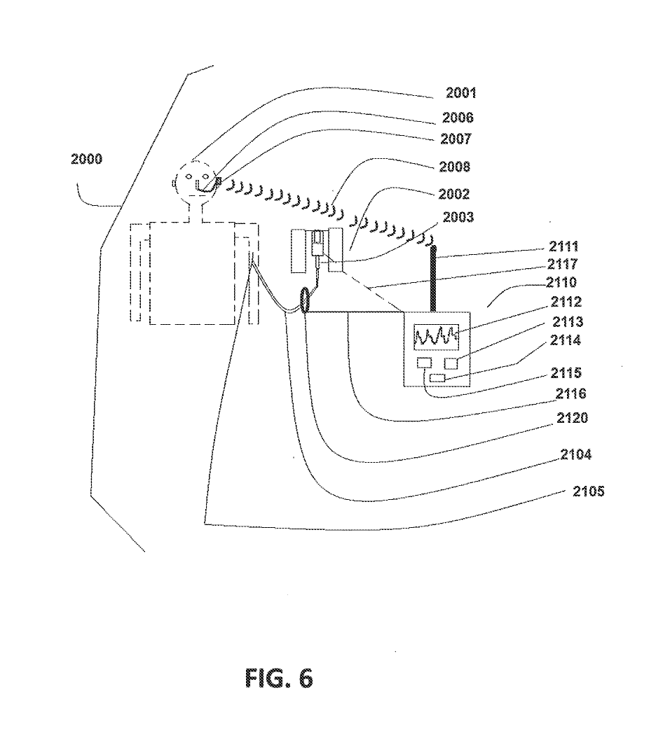

FIG. 6 depicts a particular embodiment whereby a patient is infused and an occluding device is used to decrease or stop flow of the medication to the patient. Referring now to FIG. 6, there is shown the system 2000 according to this invention in place with a subject 2001 undergoing infusion via an infusion system 2002 of a medication 2003 via, in the embodiment shown in this figure, an intravenous tubing 2104 into a vein 2105 of the subject 2001. The subject 2001, in this embodiment, is using a nasal alar Single Point of Contact (SPOC) array 2006. The SPOC array 2006 includes a communication wire running to, and for being affixed to the head of the subject 2001, by any appropriate means, including, but not limited to, for example, an over ear retention system 2007, to which the which the communication wire from 2006 runs. In this embodiment, the over ear retention system 2007 may also include appropriate local electronics, including, but not necessarily limited to, an accelerometer, or wired or wireless communications systems known in the art.

The SPOC array 2006, in some embodiments, acquires signal from the nasal alar of the subject 2001 and relays such signals to the over ear system 2007 for communication 2008 by that system to, either wirelessly for receipt by an antenna/receiver 2111 or via a wired connection, an external system 2110. The external system 2110 includes a PPG monitoring system, able to extract from the signal 2008 received from the SPOC array 2006 any desired signals for processing and analysis as herein described. The system 2110, for example, extracts heart rate 2113, respiratory rate 2114, and the subject's blood oxygen saturation level 2115. The external system 2110 is appropriately programmed and configured to develop from the signal 2008 acquired from the SPOC array 2006 a series of PD and/or PK parameters including, but not limited, patient/subject position; heart rate variability (HRV); measures of sympathovagal balance and input to the heart; heart rate and respiratory rate; autonomic nervous system function; pulse transit time (PTT); pulse wave velocity; endothelial dysfunction; arterial pressure wave shape and amplitude; ankle-brachial index; peripheral artery occlusion; arrhythmias; NIBP (Noninvasive Blood Pressure); and NAP/NAF.

When certain pre-defined parameters (preset values) are approached or reached (e.g., increase in expiratory phase of respiration, slowing of the respiratory rate, decrease in movement, increasing respiratory effort indicating airway obstruction), a narcotic reversal agent is administered to the subject, and the system 2110 also sends a signal via channel 2116 to a small occluding device 2120 deployed on the IV tubing 2104. Depending on the nature of the signal conveyed via channel 2116, the device 2120 is mechanically, pneumatically or by like means, actuated to pinch the tubing, thereby occluding flow, either partially or completely.

In some embodiments, simultaneous or near simultaneous to the signal for occlusion being sent from device 2110 via channel 2116 to the device 2120, the monitor 2110 containing appropriate software algorithms for detecting approach to or arrival at a parameter defined for this purpose, sounds an alarm. Depending on the particular infusion pump in use, this too, as a result, may sound an occlusion alarm. In some embodiments, in addition to sending the signal via channel 2116 to the device 120 to occlude or partially occlude the tubing 2104, the system according to this invention also may be integrated with the pump system 2002 to send a signal to said pump system to either turn off or slow down its rate of medication delivery. This, of course, is only possible in the subset of instances where the external PPG monitor 2110 and the pump system 2002 have compatible hardware, software and/or signals between the two which permits this direct control of the pump 2002 via the PPG system 2110.

As there already exists a large number of infusion pumps in use in a wide variety of medical care contexts, it would be a major undertaking to put in place appropriate external monitors, such as the PPG monitor 2110 according to this invention to achieve adequate and reliable communication with all the different varieties of such pumps 2002. However, the present “agnostic” system permits the system according to embodiments of this invention to be very quickly put into use in the field, in a wide variety of health-care contexts where such pumps are already in use, and to thereby provide an enhanced safety system by, on detection of an alarm condition, simply occluding or partially occluding the feed line 2104 from the pump to the subject 2001.

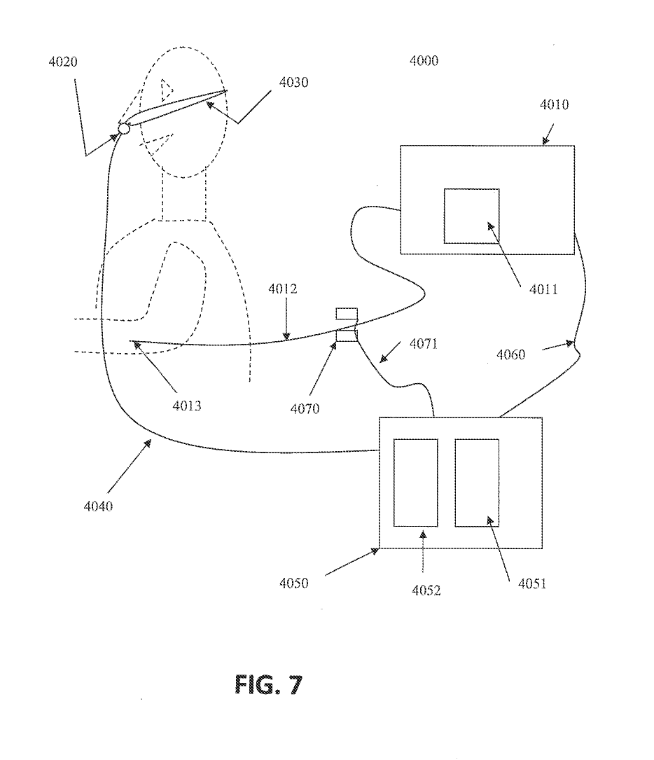

FIG. 7 provides a schematic representation of a similar embodiment of the invention, but this embodiment further includes automatically providing ventilation to a subject on detection of reduced breathing rate, increased breathing effort or both. Referring now in detail to FIG. 7, there is shown a system and apparatus 4000 in which there is provided an infusion pump 4010 for administering a CNS depressant 4011. The pump 4010 infuses the CNS depressant 4011 into a subject via an infusion line 4012 and into, for example, the arm of the subject 4013. Operatively adhered to the subject is a SPOC apparatus 4020, which may include a means for delivery of gas and for measuring expired gas, (e.g. for ETCO₂). Line 4040 includes a plurality of separate leads and hoses, including power leads to power the SPOC apparatus at the subject's nasal alar. It also includes a hose for delivery of positive pressure ventilation where such intervention is initiated by detection of hypoventilation as described herein. Line 4040 also includes signal carrying lines (or if the SPOC apparatus secured to the subject has wireless transmission capabilities, such wired communication lines may not be required), to carry the acquired signal back to the control unit 4050. The control unit 4050 is operatively connected via lead 4060 to the infusion pump for control thereof to initiate, terminate, increase or decrease infusion, based on signals acquired from the subject, including from the CSS PPG monitor.

If, however, the pump 4010 and the controller 4050 do not have compatible communication protocols, the control unit 4050 can, in any event, control infusion to the subject via the pump agnostic occluder, 4070, which, based on status of the subject, may be activated to occlude or de-occlude the line 4012 carrying infusate to the subject. Control unit 4050 includes or controls a separate source of gas 4051 for providing positive pressure ventilation to the subject when this is determined to be required by a processor unit 4052, which is pre-programmed to process the signal from the PPG sensor, and any other subject associated monitors. On determining that the subject is hypoventilating, the controller 4052 initiates the routine shown in FIG. 2. Because the SPOC apparatus at the subject is acquiring signal from which evidence of hypoventilation is derivable, it may be preferable to have the subject spontaneously breathing, without supplemental oxygen, for as much of the procedure as possible.

The methods described herein may be performed on any suitable subject, including mammals such as humans. In general, any patient that is being administered a CNS depressant for which a narcotic reversal agent exists may benefit from the methods described herein. As such, suitable environments for practicing the methods described herein include, but are not limited to, hospitals, hospices, homes, nursing homes, skilled nursing facilities, surgery centers, medical trauma settings (trauma zones, hospitals, medevac settings and the like), hiking, mountaineering, aeronautical, outer space or subaquatic environments.

Particular systems for practicing the aforementioned methods will now be described. Such systems include a PPG sensor configured to secure to a central source site of an individual; a device configured to administer a narcotic reversal agent; optionally, a device configured to administer a CNS depressant to the individual; and a controller configured (1) to receive and process PPG signals from the PPG sensor, and (2) to direct the device to administer the narcotic reversal agent to the individual if the PPG signals or a physiological parameter derived therefrom are outside a preset range of values. In some embodiments, the system may also include at least one additional sensor configured to secure to the individual.

The PPG sensors and parameters derived therefrom, additional sensors and parameters derived therefrom, central source sites, preset value ranges, CNS depressants and narcotic reversal agents have been described above. Systems and methods for operating them, according to some embodiments of the invention, have also been described above (see, e.g., FIGS. 6 and 7). However, additional information regarding the systems and methods of operation will now be described.

The systems described herein utilize a “controller” to receive and process PPG signals, and signals from other sensors, and to direct the administration of medications. As used herein, the term “controller” is meant to refer to one or more computers, microprocessors, or processing units (which may work together or independently) that receives signals from one or more PPG or other sensors operatively coupled (“secured”) to an individual and which outputs signals, at a minimum, to a device configured to administer a narcotic reversal agent to the individual. The controller may include an interface unit that includes a microprocessor and a user interface adapted to provide an interface with a user.

The controller may use only the PPG signals to determine the appropriate output signal or a plurality of sensors may be used and the algorithms may evaluate a multitude of parameters to assess the combined effects of clinical interventions and a patient's underlying clinical condition on the cardio-respiratory systems, and use this information to determine whether to administer the narcotic reversal agent.

In some embodiments of the invention, a controller may link a series of apparatuses to measure relevant PD, PK, or both PD and PK parameters of a subject, process the parameters and, on that basis, control one or more infusion pumps (cease, increase, decrease or maintain given level of infusion) for closed-loop or open-loop administration of opioids and/or other CNS depressants, and when indicated, narcotic reversal agents. In some embodiments, control of the pump or administration of narcotic reversal agents may be instantaneous or substantially instantaneous (i.e., within a few seconds or milliseconds from the acquisition of signals from the subject).

Signal acquisition from the subject may be initiated manually, or signal acquisition may be initiated automatically, for example, as a result of accelerometer signals to the control unit indicating a change in subject status, including, but not limited to, a beyond threshold period of inactivity, excessive, repetitive shaking, indicative of seizure, rapid change in vertical to horizontal orientation, indicative of a fall, or other pre-determined motion-related parameters. Of course, other motion sensing-means besides an accelerometer may be utilized for this purpose.

In some embodiments, the controller is configured to provide an open loop modality. The data from the PPG sensors and additional sensors may not be directly used to regulate the drug output from an infusion device, or to dispense the narcotic reversal agent, but may rather informs a health care worker, family member, or the patient that his/her dose requires change or no change and/or that a narcotic reversal agent may be necessary. Additionally, a “clinical advisor” system can be developed wherein a healthcare worker is notified and prompted to make appropriate changes. Thus, this is similar to a closed-loop system with algorithms analyzing the inputs from the patient and controlling the outputs from devices such as infusion pumps and non-invasive positive pressure ventilation, but rather than “closing the loop”, it alerts a healthcare worker to make the appropriate changes.

In some embodiments, the controller is configured to provide a closed loop modality. Thus, the controller (which, again, may include a number of interconnected or independent processors) may process the signals from the PPG and optionally other sensors, determine whether the specified signals or parameters are outside a preset value range, and direct the decreasing or terminating of the administration of the CNS depressant, and the initiation or increase in administration of the narcotic reversal agent, without the need for external input.

In particular embodiments, when a patient begins to have diminished cognitive and/or brainstem function, the microprocessor/controller determines, from derived parameters that the patient is beginning to have diminished responsiveness based on the characteristic changes. These are seen in the respiratory pattern, rate and depth of breathing as well as in the cardiac system, where loss of pulse rate variability is often seen. Additionally, the accelerometer determines that the patient's activity has decreased substantially, indicating that the patient is sleeping and/or suffering the effects of brainstem depression. Algorithms based on derived data may determine the differences between normal sleep and respiratory/cerebral depression. When the microprocessor determines the decreased activity and/or the derived parameters indicate respiratory depression, an alert function, such as alarms, and messages sent to care givers, family members and healthcare professional including EMS, may also be activated. This alert can be sent by conventional telephone modem, wirelessly, by cable or other means (such as satellite) to provide the necessary support for the patient.

While the methods and systems described herein are typically used in a hospital, outpatient or nursing home setting, they may also be used in other less conventional settings, such as when an individual is in an isolated environment, e.g., hiking, mountain climbing, aircraft piloting, or in a hostile environment. Thus, in some embodiments of the invention, the systems described herein may be portable, and in some cases, partially or completely wearable by an individual. In situations where medical care is not readily at hand and where a life-threatening condition arises, a wearable and/or portable narcotic administration system may be desirable. The present invention provides a substantially automated solution for evaluation of a plethora of PD and/or PK parameters of the individual and determines if they are outside present values, and if so, initiates emergency delivery of appropriate medications, fluids and the like, including narcotic reversal agents, until trained medical personnel can reach the individual and intervene if necessary. This portable system will be referred to below as a trauma environment treatment (TET) ensemble.

In some embodiments, the TET ensemble may be entirely autonomous and self-contained and all signal acquisition, processing and infusion responses may be integrated into a system which the subject incorporates into their attire (such as, for example, as part of a helmet, belt, probes affixed to appropriate physiological aspects such as nasal alae, ears and/or cheek). In some embodiments, the complete TET ensemble adds only a small fraction to the weight (normally 60-80 pounds) carried by the subject. In addition, by incorporating into TET a global positioning system, (GPS), a subject in need can be located, triaged, monitored, and optimally treated with drugs and/or fluids, either locally or remotely.

The controller may also be attached to the devices for medication administration or may be separately portable and/or wearable. Alternatively, or in addition, via appropriate telemetry and/or wired or wireless technology (whether using GPS signals, internet, 3G, 4G, infrared, ultrasound, or any other electromagnetic radiation means, now known or hereinafter developed), the system may communicate with and optionally be under the control of external analysis and/or control. This latter option provides for force-multipliers to come into operation, allowing a central entity to analyze data relevant to one or multiple individuals and to over-ride autonomous operation and provide even more appropriate interventions then are possible under completely autonomous operation of the system, method or apparatus of this invention.

The TET system, method and apparatus allows individuals to begin administration of opioids or other CNS depressants (and, if necessary, narcotic reversal agents), fluids and if necessary other medications to reduce blood loss, tolerate blood loss and/or decrease the extent of traumatic brain injury (TBI) and post traumatic stress disorder (PTSD). For the TET system, the CNS depressants, narcotic reversal agent and any other medications may be administered as described above, including intravenously, intraperitoneally, intranasally (whether in the form of a fluid, a mist, an aerosol, and/or a non-aerosol fluid delivery system and whether including or not including pharmacologically active compounds), as appropriate in a given context.

In portable systems, the delivery of fluids and/or gasses may be via appropriate pumps, or, in particular embodiments, pressurized vessels containing appropriate fluids, drugs, nutrients (e.g., glucose) and the like, which may be released in pre-metered doses on actuation of a release mechanism (a valve, servo, septum or the like). For example, each time a particular pressurized vessel is instructed by the system to release a pre-metered dose, an appropriate dose may be delivered to the subject. By sending multiple instructions, multiple doses may be applied to the subject to simulate almost continuous infusion until a reduce delivery signal or a cease delivery signal is applied to prevent further infusion of the particular agent or agents to the subject.

For intranasal delivery, the therapeutic agents could be stored in various locations of the system, including near (or in) the nose or at sites more distant from the nose (e.g., adjacent to ear or forehead). Multiple studies have shown that the nasal epithelium absorbs about 60-80% of the dose of an IV injection of the same quantity of medication. This will likely be true even if a subject is hypotensive since this area of the nasal septum is richly supplied by arteries which are branches of both the internal and external carotid. Likewise, vasopressin (unlike alpha adrenergic vasopressors) is unlikely to cause intense local vasoconstriction in the nasal area, thus allowing absorption of other medications given at the same site.

For oral medication(s), the patient may be provided with a small microprocessor/microcomputer, for example, one that is worn on the belt (or over the ear similar to a hearing aid) and attaches (either directly or by communications such as Bluetooth) to a small sensor array which is attached at a single point of contact (SPOC) array to one nasal ala. In some embodiments, the SPOC array may include one or more of the following: a pulse oximeter sensor (photodiodes [e.g., one or more LEDs] and a photodetector), a nasal pressure sensor, one of at least two ECG leads and a nasal flow sensor (thermistor or other). In some cases, the SPOC is light weight and barely visible. The SPOC array may continuously monitor cardiorespiratory parameters such as ECG, SpO₂, PPG signals (from which respiratory rate, respiratory effort, arterial blood flow, venous capacitance and other parameters are derived) and nasal pressure or flow. The SPOC system may optionally also includes an accelerometer to monitor the position of the patient.

An accelerometer or like motion and/or orientation detection sensor may be particularly useful in the TET system because it may be used to monitor whether a subject is actively moving or has suddenly ceased to move. In some cases, the accelerometer or like motion sensor is used to limit the power consumption of the TET system by maintaining it in “sleep” mode until it senses a sudden change in the subject's level of activity. In one embodiment, the accelerometer is adapted to detect very regular but intense body movement indicative of seizure activity, in which case a signal from the accelerometer sensor is processed by the controller to provide a benzodiazepine or other antiseizure medications if the subject system is in place or once the SPOC assembly is emplaced by other personnel.

The accelerometer may also be capable of monitoring the body position of the subject. A long period of inactivity in the prone or supine position is optionally programmed into the system to trigger a remote alarm so that other personnel are alerted to determine the status of the subject being monitored. Likewise, the accelerometer or other motion sensor may be used as an additional monitoring parameter while a subject is being treated by the TET system. A sudden reduction in movement is optionally programmed into the controller as an indication of inadequate pain control in the setting of acceptable vital sign parameters, while a reduction in movement coupled with unacceptable vital signs is optionally programmed into the controller to be interpreted as an urgency requiring provision of resuscitative measures, including administration of a narcotic reversal agent. In some instances, the accelerometer or alternate motion sensing component of the TET system may provide an indication of a problem with a subject, in some instances, even prior to the emplacement of SPOC on the subject—provided the subject is carrying the system somewhere in his/her kit.

The TET system may optionally remain in place as the subject is transferred to higher levels of medical care for both monitoring and drug therapy. Once IV access is obtained, drug delivery can be switched to this route. The TET may also remain in place through all levels of medical care and it may be adapted to interface with other medical treatment and monitoring systems. As such, the TET system may be adapted to provide both the initial monitoring and medication delivery to the injured subject and then continue to provide monitoring as well as medication delivery by conventional routes once IV access is obtained.

In particular embodiments, an injured subject who is conscious is able to rapidly emplace the TET on his/her nose or other appropriate site on the subject and the system immediately activates and begins providing pain medication and other medications based on the sensor data interpretation and algorithms. If the injured subject is incapacitated, a fellow subject can emplace the SPOC system on the subject. Additionally, since each subject preferably carries medications adapted for insertion into the TET system, they could be used on a wounded subject, thus increasing the amount of medication available in the field. Alternatively, or in addition, the TET assembly may be an integral part of a helmet and/or telemetry gear.

### EXAMPLES

### Example 1

### Deriving Respiratory Parameters from PPG Signals

A subject was fitted with a nasal photoplethysmography unit and a nasal pressure transducer unit. Raw data from the photoplethysmography (PPG) sensor and the nasal pressure sensor were acquired and processed as described below to derive the subject's heart rate, breath rate, and obstruction level information. These parameters are then used to govern pump titration rate and may be used to determine when to administer a narcotic reversal agent.

### DEFINITIONS, ACRONYMS, AND ABBREVIATIONS

DC=The low frequency component of either the red or infrared channels of the PPG sensor found by subtracting the AC component from the raw signal.
AC=The cardiac or high frequency component of either the red or infrared channels of the PPG sensor

### Algorithm Description

The algorithm can be broken up into three main phases: (2) filtering and preprocessing, whereby streaming data is separated into the channels that will be used in parameter calculation and individual breaths and heart beats are identified and marked; (2) parameter calculation, whereby the main predictive elements of the model are computed; and (3) model output generation, whereby the parameters are combined into the desired outputs

(1) Filtering and Preprocessing

Here the IR and RED channels of the PPG signal are first sorted into AC and DC channels using an algorithm. Whereas a standard low pass filter is typically used to separate the DC component from the raw PPG signal, this device uses the following unique approach:

1. An initial guess of heart rate (such as 60 beats per minute) is used at the onset of processing.

2. This heart rate is converted into an appropriate search window (such as 1.5/(heart rate)).

3. A local maximum is found in the raw PPG signal within this search window. This is the peak of a single heart beat.

4. A new estimate of heart rate is found by subtracting the time of previous maximum from the current maximum. This new estimate of heart rate is typically averaged with previous heart rate estimates for stability.

5. The “valleys” are found by finding the minimum value of the raw PPG signal between the current maximum and the previous maximum.

6. If there is more data, return to step #2 and repeat.

Using this approach, the locations of the peaks and valleys for each heart beat are identified and stored in a table. Halfway between each peak and valley a “midpoint” is identified. The DC component is then found by a linear interpolation between these midpoints. This approach is different from traditional approaches to finding the DC component in that it produces an estimate that does not have a lag or time shift relative to the raw PPG signal. Rapid changes in DC baseline are, therefore, more accurately captured using this approach.

The AC component is then found using a point-by-point subtraction of the DC component from the raw PPG signal. Next, the DC component is filtered using a band-pass Butterworth filter to find the respiratory component of the PPG signal. Two possible ways the band-pass cutoff frequencies can be determined are:

1. Use a set range based on common breath rates (such as 1 to 0.1 Hz); and

2. Use the nasal pressure signal to determine the average breath rate and then center the filter cutoffs over that breath rate.

The nasal pressure signal is then also filtered using a band-pass Butterworth filter to remove artifacts and noise. Filtering the nasal pressure signal helps identify prominent breath features (peak inhalation, peak exhalation, etc) and helps reject noise and motion artifacts. Finally the individual breaths are identified in the pressure signal. The start-of-inspiration (SOI) and end-of-breath (EOB) as well as the peak inhalation and exhalation are found and stored in a table.

(2) Parameter Calculation

From the nasal pressure and two PPG channels (IR and RED) a wide range of parameters can be calculated to help predict respiratory and cardiac phenomena. Some of these parameters include:
Nasal Pressure Amplitude: the distance between the peak of inhalation and the peak of exhalation for each breath averaged within a time window (1 minute for instance);
Nasal Pressure Breath Rate: The average breath rate found within a window of time;
Nasal Pressure Amplitude Variance: the variance of all the nasal pressure amplitudes found within a time window;
Nasal Pressure Breath Period Variance: the variance of the individual breath times (end-of-breath time minus start-of-breath time) for each breath within a time window;
DC Drop: the distance between the base of a DC drop and its baseline (baseline is typically the average DC value over a larger time window);
DC Drop Duration: the time it takes for the DC component to return to baseline after a drop from baseline;
DC Drop Area: the area found by integrating the signal (DC Baseline-DC Component) during a DC drop from baseline;
AC Heart Rate: the average heart rate found in the AC component within a time window;
AC Heart Period Variance: the variance of the individual heart beat lengths within a time window;
AC Amplitude: an average of the individual heart beat amplitudes (maximum minus minimum) within a time window;
AC Amplitude Variance: the variance of the individual heart beat amplitudes within a time window;
SAO2 Drop: the drop in the blood O₂ saturation found by converting the IR and RED PPG signals into an estimate of blood oxygenation (ie the more traditional use of the PPG signals); and
PPG Resp Energy: the energy in the respiratory component of the PPG signal within a time window.

(3) Model Output Generation

The parameters described above are typically converted into unit-less “percent” values. This is done by calculating a baseline using a large time window and then each parameter is converted to a percent-change-from-baseline. After this conversion, the parameters are then combined in appropriate proportions to generate model outputs. Most commonly, these parameters are combined using a simple linear combination though a more advanced method such as tap-delay lines or neural networks can also be used.

The parameters described above can be combined to produce signals that regulate the titration of the infusion pump and can be used to determine when to administer a narcotic reversal agent. The two main model outputs that control the pump are “Breath Rate” and “Obstruction Level”. Other indications of respiratory or cardiac distress can also be inferred from these parameters and pump infusion rate (or rate of narcotic reversal agent) can be adjusted accordingly.

Based on the processing of the PPG and nasal pressure signals, the system of this invention is able to select which drugs, and the quantities of such drugs to be administered to the subject, and to aid in determining when a narcotic reversal agent should be administered. Of course, ongoing iterative application of given pharmacologic and fluidic interventions are reflected in the ongoing monitoring of PD, PK or PD and PK parameters acquired from the subject, allowing for dynamic modifications to the intervention, within appropriate pre-set limits defined by qualified medical personnel for a given context.

### Example 2

### Graphical User Interface of Infusion Monitor

A closed-loop or open loop system or apparatus may be emplaced on a subject, either by the subject or a colleague, physician, or the like. On being emplaced, the system initiates, conducts an internal self check to ensure that it is operating properly, that it has sufficient power for reliable operation, that it is properly interfaced with the subject and is able to acquire appropriate PD, PK, or PD and PK signals from the subject. The thus emplaced and properly operational system may then be used to monitor the subject or it may go into a sleep or standby mode in which operational parameters are minimized along with minimal power consumption.

On being stimulated by an appropriate wake-up signal, which may be the subject pressing a start button, or an integrated motion sensor such as an accelerometer recognizing a motion state that is defined as requiring wake-up (e.g., excessive vibration, or no motion at all by the subject, or a sudden change in vertical to horizontal orientation), or due to an external telemetry signal from a central monitoring station, the system wakes up, quickly performs an operational self check and then measures appropriate PD and/or PK or other parameters for the subject. If all parameters check out as being normal or within pre-defined acceptable tolerances, the unit may once again enter a sleep mode. If any parameters are out of pre-defined tolerance, the unit immediately initiates delivery to the subject appropriate agents (fluids and/or nutrients, pharmacologically active agents or narcotic reversal agent), to bring the subject's parameters back within pre-defined acceptable tolerances (“preset values”). The unit may be entirely self-contained and autonomous and may require little or no intervention from the subject themselves or from external personnel.

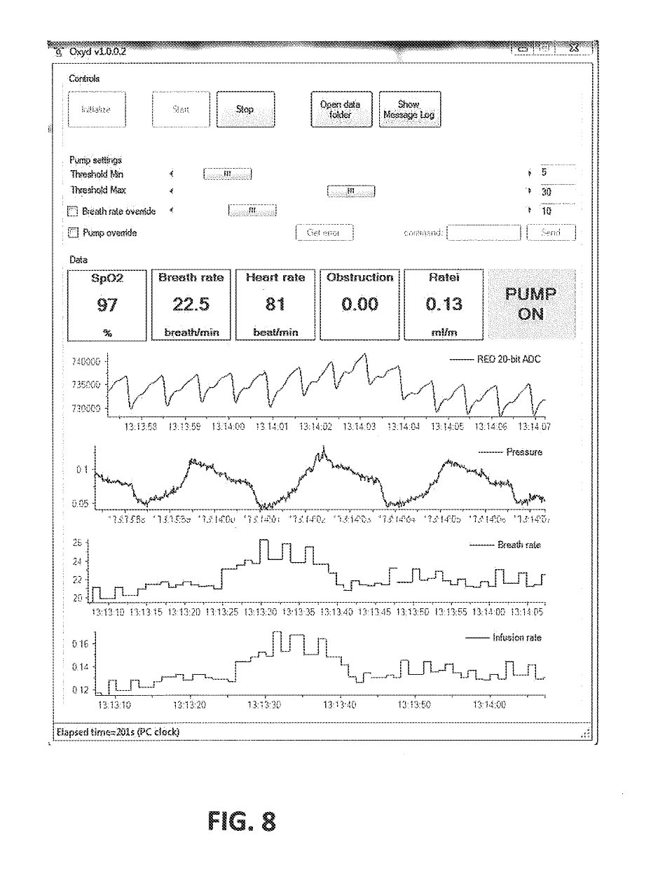

In an operational prototype of the present invention, a graphical user interface is provided, shown in FIG. 8. This is not intended to limit the interface options that are available in the apparatus or system of the invention. Rather, this is intended only to show that an operational monitor has been achieved, and to provide an example of a user interface. Turning to FIG. 8, it the following elements can be seen and are understood as follows:
At the top of the figure, a variety of settings for the pump control software are shown, including the minimum and maximum thresholds that determine when the pump is fully on and when it is fully off. There is an override for the pump and breath rate to permit manually setting the pump or the breath rate.
Numeric values are shown for breath rate, heart rate, and “rater” Ratei is the current infusion pump setting (rate of infusion), which changes with breath rate or other cardiorespiratory parameters (e.g., respiratory effort, heart rate, arrhythmias), and an indicator that the pump is currently on.
There are two raw signals from the pulse-oximeter, infrared and red that are used in combination to determine the oxygen saturation (SpO2). The IR signal is less sensitive to saturation changes and thus provides a more stable signal for PPG processing for purposes of this invention.
The nasal pressure indicates the change in pressure in the nasal opening during breathing. AIN is analog input 0 from the A/D converter, which is obtained from the pressure sensor. This signal very accurately represents breathing, including when mouth breathing is occurring.
The first two graphs show the real-time breathing and pulse. The next two graphs show breath rate and infusion rate, and illustrate how the infusion rate changes over time based on the measured breath rate.
The Red 20 bit ADC value is obtained via an OxyPleth pulse oximeter. In practice, this would be the value coming directly off the photodetector when the red LED is pulsing, (typically, pulse oximeters pulse red and infrared light alternatively into a single photodetector). Both signals are obtained by the PC via the serial port of the OxyPleth.
The nasal pressure signal is obtained through a nasal oxygen canula and is converted via a very sensitive pressure transducer (Microswitch, part #DCXL01DS) and then A/D converted via an A/D converter.
The breath rate is calculated from the nasal pressure signal by detecting changes in pressure during the breathing signal, or alternatively can be calculated via changes in the PPG signal.
The infusion rate signal is sent to the infusion pump to dynamically control it. Currently, this signal is derived from the breath signal (which comes from the nasal pressure signal, but could also come from the pleth/IR signal). When the breath rate is high, the pump is on fully. When the breath rate falls below the upper threshold, the pump rate decreases until the lower threshold, at which point it turns off. This represents one simple method of controlling the pump. There are much more sophisticated ways in which those skilled in the art could modify this, based on the present disclosure, including, but not limited to, by using breathing pattern characteristics, such as entropy of the breathing pattern, and the like.

### Example 3

### Detection of Respiratory Events with PPG and PSG

Polysomnography (PSG) and PPG data was obtained from 35 subjects and scored manually by a trained research technician. The data on the first 20 subjects will be used as a training set, and the data on the remaining 15 subjects used as a validation set. Optionally, a study to collect data on up to 10 subjects with epiglottic catheter as a measure of respiratory effort was included.

Preliminary assessment of the prototype AHI estimator based on new patient data and analysis/integration of appropriate algorithms and analysis is provided summarizing in-sample data. To determine the accuracy of the SPCDS, RDIs were calculated for each study and compared to manual scoring. Receiver-operator characteristic curves can be constructed for the RDIs calculated to assess the performance of the automated algorithm across the spectrum of SDB severity (RDI cutoffs of 5, 10, 15, 20 and 30 events per hour for defining obstructive sleep apnea). The area under the receiver-operator characteristic curve were calculated for each threshold and reported with the standard error and the limits of the 95% confidence interval. Positive likelihood ratio, negative likelihood ratio, optimum sensitivity and specificity were calculated for each threshold. An epoch by epoch assessment of agreement for the detection of respiratory events was conducted. The outcome of this work was the development of a prototype algorithm validated on 20 subjects recruited from a sleep lab. The operation of the prototype was validated using analysis of a 15 patient test set utilizing the statistical methods described above and below.

There are three types of synchronization that we implemented during this project. First, low level synchronization involves the alignment of the pulse-oximetry/photoplethysmography (PPG) data with the polysomnography (PSG) data. Second, to optimally detect events, a portion of the parameters that are delayed indicators of events (e.g., post-event parameters) must be “aligned” with the parameters that are already synchronized with the events. And third, “predicted event to scored event” synchronization to allow for the matching of SPOC-labeled events with manually scored events is necessary to determine sensitivity and specificity values.

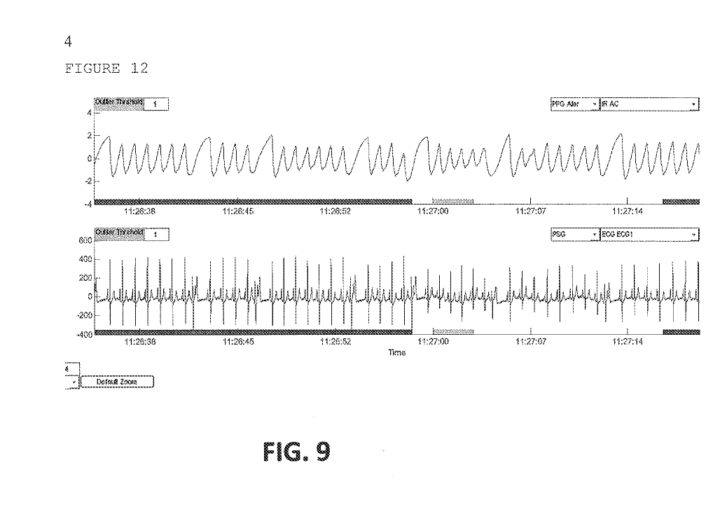

The PSG data is collected via the Alice system and the PPG data is collected using a NICO monitor connected to a PC utilizing a LabView program. The LabView program sends the PPG data along with sync pulses to the Alice system to ensure that the data remains aligned. Unfortunately, the data typically slowly drifted out of alignment, even when using the sync pulses. The sync pulses only ended up providing a rough but inaccurate alignment of the data. We utilized a genetic alignment algorithm to match the two data streams by maximizing the correlation between the ECG channel in the PSG and the AC signal in the PPG. The results for each patient were validated manually and the alignment was determined to be excellent. An example alignment is shown in FIG. 9.

The second synchronization effort is one of aligning parameters that correspond to events with parameters that correspond to post-event phenomena. For instance, the nasal pressure signal drops during an apnea event, but the pleth DC signal drops during the post-event time. In order to maximize the classification capability of these signals, it is desirable to shift the pleth DC signal back in time to be better aligned with the nasal pressure signal. To optimize this process, we determined the maximum area under the curve (AUC) of each parameter's event-prediction ROC curve. We then shifted the parameters and determined the shift that produced the largest AUC (e.g., the best prediction). This synchronization dramatically increased the discrimination provided by these “post-event” parameters.

The third synchronization, aligning the predicted and actual events for sensitivity analysis, will be described in greater detail in the Results section. To derive a predictive model, there are multiple levels of optimization that can be utilized. First, individual parameters must be conceived, implemented, evaluated, and optimized. Second, individual parameters must be combined optimally to create the desired model.

The first step in creating a model to detect events is to create appropriate parameters that capture information of interest. Once the physiologic effects are identified, parameters are coded and evaluated to determine how well they capture the information intended and how well the information predicts the events. Each physiologic effect (e.g., venous capacitance change, reflected by a change in pleth DC value) may have several possible parameters that attempt to capture its useful information (e.g., area in the DC drop, DC drop depth, DC drop time, etc.) and each parameter may have several sub-parameters that need to be optimized (e.g., window width to determine DC baseline for calculating DC drop). All of these parameters and sub-parameters were optimized using the AUC of an ROC curve generated by separating event breaths from non-event breaths. This AUC methodology allowed us to optimize the individual parameters without having to do end-to-end comparisons of event detection (e.g., event synchronization, RDI calculation, etc.). The AUC methodology provides a method of maximizing each parameter's ability to separate the event vs. non-event distributions.

The physiologic effects we attempted to parameterize were:
Venous Compartmentalization
   Rise of DC during events
   Fall of DC during arousals
   Slope of DC “recovery”
   Envelope changes in the BR signal.
Saturation:
   Drop/Rise in SpO₂ over IR during event/recovery.
   Desaturation slope
Respiratory System:
   Amplitude of flow and pressure drops/rises during events/arousals.
   Breath Amplitude variability
   Shark fin pattern during early part of occlusion
   Breathing effort pattern from IRDC curve.
Cardiac System:
   HR & HR variability
   AC amplitude and AC amplitude variance
Nervous system:
   HR variability, Breath Rate variability, IR DC variability

Because many of the parameters are based on characteristics of breathing, we first parsed the data files into breaths to allow for a consistent methodology for parameterization and averaging. Breaths were determined based on the nasal pressure signal. During apneas when the breathing was not easily determined, an average breath rate was utilized to parse the data. The training set was then labeled from the manual scoring table, producing breath-by-breath labeling of the events. Each parameter was then calculated for each breath and the breath-based labeling and parameters were used to calculate ROC curves. Breath-by-breath analysis is not optimal since an event might be 3-5 breaths and a parameter might miss the first and last breath, for instance. This technique, however, does provide a low-complexity methodology for determining the separation provided by the parameters and allows for optimization of the parameters and sub-parameters.

The parameters derived from this analysis consist of:
5 Nasal pressure parameters
6 SpO₂ parameters
9 Pleth cardiac parameters
8 Pleth low frequency parameters
3 Pleth breath parameters (bandpass filtered at breath rate)

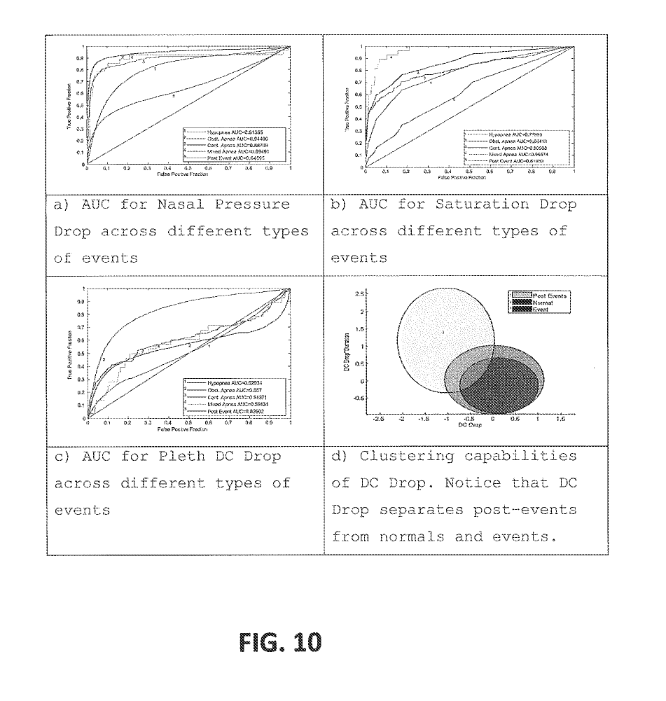

FIG. 10 shows several plots indicating the performance of the individual parameters on breath-by-breath classification. Once the individual parameters are optimized, the next step is to create multi-parameter models that maximally capture the information and coupling of the individual parameters as well as the temporal structure of the data. An important consideration in multi-parameter modeling is that it is the unique (independent of other parameters already in the model) information that a parameter adds to the model that makes it valuable, not its individual ability to separate the classes. Another important point is that optimization of any model requires good criteria. We determined that the best result is one that maximizes multiple criteria simultaneously: correlation with RDI, Kappa statistic for epoch-by-epoch confusion matrices, and diagnostic agreement. Although this complicates the optimization process, the performance surfaces of the models was not steep or highly non-linear, so optimization of multiple criteria was possible without excessive effort.

To use these statistics for optimization, however, we needed to implement several algorithms to compute them. First, events were predicted by the multi-parameter model and a windowing algorithm was used to modify breath-by-breath events into events similar to those scored manually (e.g., 10 second events, etc.). The RDI was calculated by summing the events and dividing by “valid study time” (note: not sleep time). The epoch-by-epoch confusion matrices were computed by summing the predicted and scored events per 30 second epoch. Diagnostic agreement was also computed based on the ability of the system to accurately predict a range of RDIs (more information in the Results section). Some subtleties exist in these statistics. For instance, high RDI patients may have 1000s of events whereas low RDI patients may have 10s of events. The high RDI patients will therefore dominate the epoch-by-epoch Kappa value.

An important feature of our multi-parameter modelling is the addition of temporal information. Many of the parameters are highly predictive of events, but have a high rate of false positives as well. When analyzing the data however, it is clear that events have a different temporal structure (smooth) than the false alarms (peaky). In addition, some parameters detect events, some parameters predict recovery (or post-events), and some parameters indicate normal breathing. By utilizing a temporal model, additional information about the progression of the signals over time can be utilized to make decisions.

There are many approaches to adding temporal information. The most common approach is averaging which is a subset of moving average filters (finite impulse response filters, or FIRs). Strict averaging multiplies each sample by 1/N (where N is the number of samples in the average) and sums the results. Moving average or FIR filters are similar, except that each sample can have a different weight. This allows the filter to give varying emphasis to different delays or time frames (for instance, more emphasis to the recent past than the distant past). Implementation of this type of filter often includes the concept of a tap-delay line which is a memory structure that stores the recent past of the signal and scales each one to create the model output. We call this approach the TDL (tap-delay line) and use it as our baseline temporal filtering approach.

We also experimented with temporal neural network models and the Hidden Markov Model (HMM). We utilized a tap-delay neural network (TDNN) model which is the most common temporal neural network and is a non-linear generalization of the FIR filter. The HMM provides a state-based (stochastic) approach to extracting temporal information. The HMM creates states based on the inputs to the model and calculates the likelihood that the current set of data was generated by the model. Therefore, an HMM model would be created with apnea events and the data leading up to and following the event. Other HMM models would be created to represent other events or normal breathing. New data is passed through all the models and the model that has the highest probability of matching the data “labels” the data.

In this study, with only 20 patients in the training set, the TDL, TDNN, and HMM models all produced roughly equivalent performance. In modeling theory, the simplest model that has adequate performance is most likely to generalize across new data, particularly with a small training set (increased complexity requires larger training sets to adequately train). For this reason, our analysis focused on the TDL model. Experimentally, 5 memory elements were sufficient to capture the information of interest in the signal. Typically, this memory was centered on the breath of interest, meaning that the memory structure contained the breath under test and the 2 breaths before and after it.

Several side-studies were implemented during the project. One such study looked at the ability of the parameters to determine arousals. In our database, 72% of events have a labeled arousal within 5 seconds after the event. The majority of the remaining 28% appear to have similar characteristics to an arousal in the breathing parameters, but are not labeled as arousals (possibly due to insufficient EEG activity). In a quick evaluation of our parameters, we were able to detect these arousals using only DC drop with an AUC of 0.85.

Another topic of interest was whether the saturation information at the central site was similar in value and discriminability to the saturation at the finger. The three studies were scored, first with the finger saturation and a month later with the nasal alar saturation. The scoring is shown in the table below. We also calculated the epoch-by-epoch confusion matrix and determined that the Kappa statistic for this matrix was 0.92 and had an agreement rate of 98%. The differences in the scoring are similar to if not less than the typical difference in scoring between multiple scorers, and thus considered insignificant.

| | | | | | Nasal Alar | | |
|---|---|---|---|---|---|---|---|
| | Finger SpO2 | Alar SpO2 | | | 0 | 1 | 2 |
| SPOC-04 | 36.5 | 36.1 | Finger | 0 | 2368 | 9 | 0 |
| SPOC-06 | 29.1 | 25.2 | | 1 | 51 | 420 | 0 |
| SPOC-08 | 13.9 | 12.2 | | 2 | 0 | 0 | 7 |

Next, we evaluated the differences in our models when nasal saturation was replaced by finger saturation. Some caveats of note are that the NICO (alar) reports saturation in increments of 1% whereas the Alice system (finger) reports saturation in increments of 0.1%. When looking for saturation drops of 2-5%, the increased resolution of the Alice system is particularly important. Additionally, the NICO does not seem to handle the increased signal strength of the ear-lobe sensor when attached to the alar. The alar has less soft tissue and more blood flow than the finger, thus producing a much stronger signal. In our previous studies using the Novametrix Oxypleth, we did not have this problem. The NICO tended to threshold the saturation at 100% and thus produced even less resolution than the finger. It is important to note that this is a data collection limitation, not a physiologic limitation. The following table shows the percent of the time that the saturation at the nasal alar was determined to be 100% (relatively uncommon normally).

| | Total | | |
|---|---|---|---|
| | Clipped | Total Record | % Time |
| Patient | Time (hrs) | Time (hrs) | Clipped |
| SPOC-01 | 3.58 | 8.75 | 40.9% |
| SPOC-02 | 5.69 | 8.77 | 64.8% |
| SPOC-03 | 2.85 | 3.37 | 84.4% |
| SPOC-04 | 0.27 | 7.40 | 3.7% |
| SPOC-05 | 0.00 | 6.76 | 0.0% |
| SPOC-06 | 0.35 | 7.80 | 4.5% |
| SPOC-07 | 1.64 | 6.62 | 24.8% |
| SPOC-08 | 0.26 | 8.79 | 3.0% |
| SPOC-09 | 0.42 | 7.21 | 5.8% |
| SPOC-10 | 0.73 | 6.06 | 12.1% |
| SPOC-11 | 0.02 | 7.70 | 0.2% |
| SPOC-12 | 7.64 | 7.83 | 97.7% |
| SPOC-13 | 4.35 | 7.53 | 57.8% |
| SPOC-14 | 3.40 | 7.85 | 43.3% |
| SPOC-16 | 1.14 | 7.86 | 14.5% |
| SPOC-17 | 0.09 | 7.20 | 1.2% |
| SPOC-18 | 0.01 | 6.91 | 0.1% |
| SPOC-19 | 4.81 | 7.34 | 65.6% |
| SPOC-20 | 0.02 | 6.40 | 0.3% |
| SPOC-21 | 0.01 | 6.23 | 0.2% |
| SPOC-22 | 2.93 | 7.79 | 37.6% |
| SPOC-23 | 4.77 | 7.96 | 59.9% |
| SPOC-24 | 1.01 | 5.34 | 18.9% |
| SPOC-25 | 0.00 | 7.13 | 0.0% |
| SPOC-26 | 0.07 | 2.96 | 2.3% |
| SPOC-27 | 2.76 | 7.07 | 39.0% |
| SPOC-28 | 1.37 | 8.49 | 16.2% |
| SPOC-29 | 0.32 | 6.52 | 4.9% |
| SPOC-30 | 1.00 | 6.43 | 15.5% |
| SPOC-31 | 1.28 | 6.64 | 19.2% |
| SPOC-33 | 0.06 | 6.63 | 0.9% |
| SPOC-34 | 0.07 | 7.56 | 0.9% |
| SPOC-35 | 0.71 | 7.35 | 9.6% |
| SPOC-36 | 0.94 | 5.20 | 18.1% |
| SPOC-37 | 3.14 | 7.26 | 43.3% |

When comparing nasal alar saturation and finger saturation, we found that the average saturation drop during events with the nasal alar was 2.5±1.8 and with the finger 2.8±2.1. When analyzing the delays in the signals by calculating the optimal time-shift to align the saturation drop with the event window, the finger saturation delay was 7.5 seconds and the nasal alar delay was 5 seconds. Theoretically, central sites may desaturate faster than peripheral sites, although this cannot be strictly proven with this data due to differences in the data acquisition of the finger (Alice) and alar (NICO). Lastly, we calculated the ROC curves for detection of events with the nasal and finger saturation. FIG. 10(b) shows that these two ROC curves are virtually identical. Thus, although the saturation signals were collected differently and were suboptimal at the nasal alar, the information content of both signals was equivalent.

To further analyze the differences in saturation, and also create baseline model statistics, we endeavored to automatically calculate the manual scoring oxygenation desaturation indices (ODIs) from the PSG and PPG data. In the patient reports, the Desat Index is simply given as “#/hr”, with no further explanation of how it is calculated. We assumed they used a 3% cutoff to get the number of Desats (#) and that they divided by Time in Bed (TIB), but we don't know if these assumptions are correct.

For our calculations, the Desaturation Index is equal to the number of times the SpO₂ value falls below a cutoff value (relative to a baseline) divided by the time in bed (TIB). For both the predicted alar-based (PPG) and finger-based (PSG) desaturation indices, we evaluated a variety of SpO₂ cutoff values to determine which one most closely matched the manually scored Desaturation Index as well as dividing by both TIB and total sleep time (TST). The TIB is the time from Light Off to Light On and TIB is equal to the TST plus the times labeled WK. We optimized these parameters by minimizing the mean squared error (MSE) between the predicted ODI and the manually scored ODI. It turns out that using the PSG SPO₂ to predict scoring (optimal possible solution), a cutoff of 3.5% and TIB gave the lowest MSE. Except for 3 patients, the difference between Total Recording time and TIB is less than 30 minutes.

From this optimization, we calculated 3 sets of Desat Indices:
Using the PSG signal, we calculated Desat Index=# of Desats/TIB (Column C) using a cutoff of 3.5%.
Using the PPG signal, we calculated Desat Index=# Desats/TIB (Column D) using a cutoff of 3.01%.
Using the PPG signal, we calculated Desat Index=# Desats/Total Recording Time (Column E) using a cutoff of 3.01%.

The results are shown in the table below. We also calculated the mean squared error without patients 16 and 18. Because these two patients have large Desat Index values, they also have larger absolute error values and have a disproportionate effect on the MSE value (L₂ and high norms emphasize larger errors more than smaller errors). We thought it would be helpful to look at the MSE without these two patients included. The table shows MSE with and without those two patients.

| | | Column C | Column D | Column E |
|---|---|---|---|---|
| | | Calculated Desat Index | | |
| Column A | Column B | PSG | PSG | PSG |
| Patient | Given Desat | cutoff = | cutoff = | cutoff = |
| (SPOC)# | Index (PSG) | 3.5%/TIB | 3.01%/TIB | 3.01%/Rectime |
| 1 | 7.4 | 7.3 | 9.0 | 9.2 |
| 2 | 3.6 | 7.2 | 4.0 | 4.2 |
| 3 | 4.7 | 2.4 | 0.9 | 0.9 |
| 4 | 14.5 | 15.6 | 15.8 | 15.5 |
| 6 | 17.9 | 20.5 | 15.9 | 16.5 |
| 8 | 7.4 | 10.4 | 7.8 | 7.5 |
| 9 | 8.9 | 6.4 | 15.5 | 15.1 |
| 11 | 1.3 | 0.0 | 0.0 | 3.8 |
| 12 | 0.1 | 0.2 | 0.0 | 0.0 |
| 13 | 7.1 | 7.1 | 5.2 | 5.0 |
| 14 | 10.1 | 9.0 | 8.9 | 8.6 |
| 16 | 94.1 | 88.0 | 80.1 | 77.1 |
| 17 | 0.6 | 2.2 | 1.6 | 1.5 |
| 18 | 39.8 | 42.1 | 33.8 | 31.4 |
| 19 | 5.1 | 3.5 | 1.0 | 0.9 |
| 20 | 20.2 | 14.8 | 14.8 | 13.9 |
| 21 | 2.0 | 7.0 | 6.2 | 3.5 |
| Mean Std. Dev. | 14.4 | 14.3 | 13.0 | 12.6 |
| | 22.7 | 21.5 | 19.3 | 18.4 |
| MSE* MSE: no | 0 | 8.6 | 21.8 | 29.0 |
| 16 & 18** | 0 | 7.0 | 9.2 | 8.8 |

*MSE: Mean Squared Error between values in column and Given Desat Index (Column B)
**MSE no 16 & 18: Mean Square Error not including patients 16 and 18 (patients with very high index values)

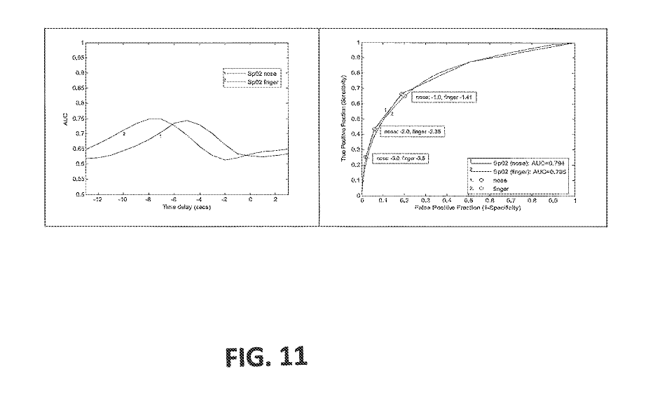

FIG. 11 shows the excellent correlation between the ODI calculated with the nasal probe and the ODI calculated with the finger probe. The correlation coefficient is 0.987 and the bias is 0.7 with a precision of 2.

We also implemented a short study to determine the ability of the current SPOC data to predict the difference between central and obstructive apneas. In particular, we studied the EPISPOC patients since the epiglottal catheter allows for more “scientific” scoring of obstructive, central, and mixed apneas. At the time this study was done, 4 EPISPOC patients were available (102-105). The study utilized a new parameter called BR Energy. BR Energy estimates the breath effort by summing the energy (square of BR signal) over a 10-second window and dividing by the average energy over a 300-second baseline window. This methodology determines changes in breathing effort. The tables below summarize the performance of the model to detect the difference between central and obstructive apnea and also the difference between central and mixed versus obstructive apnea. Agreement rates are good and the Kappa statistic indicates “moderate agreement” between the PSG and predicted labeling.

| | | | | Central and Mixed vs. Obstructive | | | |
|---|---|---|---|---|---|---|---|
| Central vs. | | | | | | CE System | |
| | | CE System | | | | Cen/ | |
| | | Central | Obst | | | Mix | Obst |
| PSG | Central | 40 | 39 | PSG | Cen/Mix | 256 | 94 |
| | Obst | 28 | 465 | | Obst | 135 | 358 |
| | | CE System | | | | CE System | |
| | | Central | Obst | | | Central | Obst |
| PSG | Central | 7.0% | 6.8% | PSG | Central | 30.4% | 11.2% |
| | Obst | 4.9% | 81.3% | | Obst | 16.0% | 42.5% |

Kappa = 0.48, Agreement = 88%
Kappa = 0.48, Agreement =

The SPOC model evolved over time to include the following parameters:
Nasal pressure drop: for each breath, the percent change in amplitude from baseline is computed. The signal is filtered to remove high-frequency spikes and outliers, and the nasal pressure drop is computed as the difference between the baseline peak amplitude minus the maximum peak amplitude during the breath. For stable breathing, the baseline peak amplitude is the average of peak amplitude over a 40-breath window centered on the breath of interest. For unstable breathing (e.g. during periods of many events), the baseline peak amplitude is the mean of the largest 50% of the peaks in that window.
SpO₂ drop: for each breath, SpO₂ Drop is computed as the mean of the SpO₂ during that breath subtracted from baseline. The baseline SpO₂ is calculated as the modified median of the SpO₂ in the two minute window centered on the current breath, where the modified median is the 80^{th} percentile value of the sorted breaths in that window.
Pleth DC drop area: for each breath, DC Drop Area is the integral of the portion of the DC signal that drops 1% or more below the baseline. The AC and DC signals are separated using the patented algorithm to optimally separate the cardiac signals from the respiratory and other signals. The baseline is computed as the average of the DC signal in a five-minute window centered on the breath of interest.
Pleth heart rate: for each breath, the pleth cardiac signal is parsed for peaks and the heart rate is determined by counting the peaks in the preceding 10 seconds.

Each of these parameters is time shifted (when necessary) and weighted using a five-tap delay line (TDL model) to create a single signal that indicates events. An optimal threshold is then determined to detect events. The events are then utilized to calculate RDI, the epoch-by-epoch Kappa statistic, and diagnostic agreement.

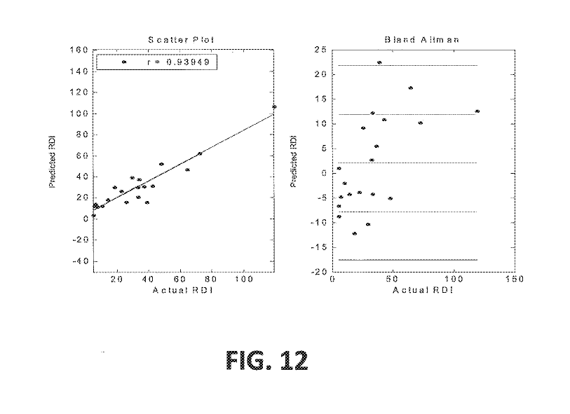

Performance of this model was good as shown in FIG. 12; it is noted that the models must be scaled to correlate well with RDI, rather than actually determining the actual value of RDI. The model may be improved through evaluation of robustness and routine experimentation.

We not only created a new model that matched RDI without scaling, we also did a series of tests on the models to determine their “robustness” and ability to generalize outside of the training set. The resulting new model performs well on mean RDI error (mean absolute error of 8.9, dominated by the large RDI patients), diagnostic agreement (95%), and the Kappa statistic of the confusion matrix (0.465). The new model replaced the “Pleth DC Drop Area” parameter with the similar “Pleth IR DC Drop” parameter and replaced the “Pleth heart rate” parameter with the “Pleth Red AC Amplitude Variance” parameter.
Pleth IR DC Drop: for each breath, the IR DC Drop is calculated as the ratio between the average IR DC value during the breath and the baseline IR DC value. The baseline IR DC value is an average of the IR DC value over a 40-second window centered on the current breath.
Pleth Red AC Amplitude Variance: for each breath, the Pleth Red AC Amplitude Variance is calculated as the variance of the peak-to-trough distances of all beats detected in the breath and 10 seconds prior to the breath.

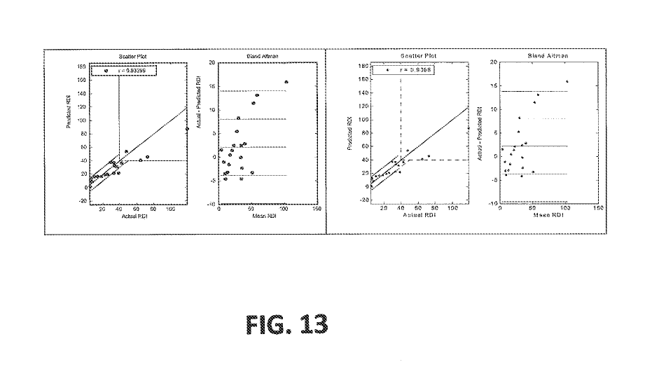

Model robustness was evaluated using the leave-one-out and leave-five-out techniques. In the leave-one-out method, 15 different models were created with only 14 of the 15 patients with RDI<40. Each model was used to only predict the RDI for the one patient not included in the training set. The final evaluation is determined by calculating statistics for the 15 different models on each of the “left out” patients. As shown in FIG. 13, performance of the model during the leave-one-out testing was nearly identical to the performance of the model using all 15 patients as the training and testing sets. This indicates that the model is robust across all 15 patients used in this study.

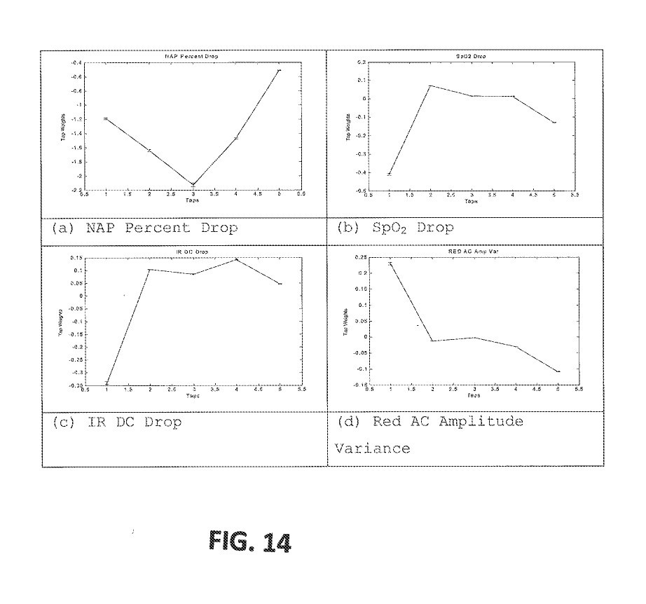

To further test the robustness of this new model, we implemented a leave-five-out methodology that utilizes only 10 patient databases for training. This is a more difficult task since the training set is smaller. Performance was similar to above again proving successful generalization. We also analyzed the variance of the weights in the model. A good model will have very similar weights when trained on different data sets—this indicates that the model is not sensitive to the choice of training set and is capturing the information of interest. FIG. 14 shows the weights for each of the 5 taps of the TDL for each parameter in the final model. In particular, notice the variance bars for each weight and how small the variance is between the 50 random selections of 10 patients. This is an excellent indication that the models are robust to patient selection.

Our last check to ensure we have a robust model is to utilize the EPISPOC patients as an independent test set. Using the 15 patients with RDI<40 as the training set and the 4 good EPISPOC patients as the test set, we achieved a correlation coefficient of 0.99 and a 100% diagnostic agreement. The table below shows the predicted and actual RDIs for these patients.

| | PSG RDI | SPOC RDI |
|---|---|---|
| EPISPOC-102 | 48.4 | 53.2 |
| EPISPOC-103 | 42.2 | 51.1 |
| EPISPOC-104 | 70.2 | 75.9 |
| EPISPOC-105 | 47.5 | 53.6 |

In summary, all indications are that this model should generalize well to new data, under the following assumptions: (1) The training data represents the population of interest well, and (2) the test data comes from the same population as the training data.

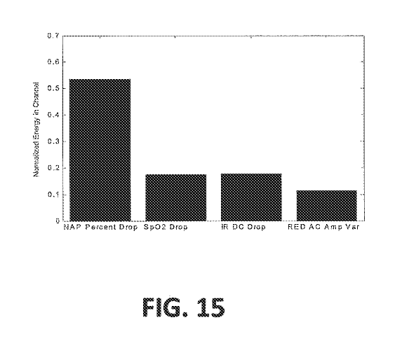

It is desirable to understand the amount of information from each parameter that is utilized by the model. To do this, the energy in each of the four channels was summed across the 20 patients and the four parameters were then normalized to sum to 1. FIG. 15 shows the contribution from each channel in the model's output. As expected, nasal pressure has the largest single contribution to the model at ˜50%, with the other three parameters contributing between 10% and 18%.

Further analysis shows that the largest errors in the prediction of the RDI arise from patients who have a significant difference between sleep time and study time. The table below shows that the two patients who fell outside the White/Westbrook diagnostic agreement both had significant wake times during the study. The current SPOC model does not have the capability to compute sleep time and therefore assumes the patient is asleep during the entire study.

| | | | TST Over- |
|---|---|---|---|
| | PSG RDI | SPOC RDI | Prediction (hrs) |
| SPOC-01 | 33.2 | 21.8 | 4.3 |
| SPOC-02 | 10.2 | 14.9 | 0.9 |
| SPOC-03 | 18 | 16.1 | −1.6 |
| SPOC-04 | 36.5 | 33.1 | 2.3 |
| SPOC-05 | 5.3 | 11.6 | 2.3 |
| SPOC-06 | 29.1 | 38.1 | 1.1 |
| SPOC-07 | 25.2 | 20.9 | 1.0 |
| SPOC-08 | 13.9 | 17.1 | 1.2 |
| SPOC-09 | 32.6 | 36.0 | 1.2 |
| SPOC-10 | 47.5 | 53.0 | 0.3 |
| SPOC-11 | 5.5 | 13.4 | 0.9 |
| SPOC-12 | 4.8 | 1.6 | 2.8 |
| SPOC-13 | 33.3 | 34.4 | 1.5 |
| SPOC-14 | 42.4 | 37.9 | 1.5 |
| SPOC-16 | 119 | 92.1 | 0.5 |
| SPOC-17 | 6.9 | 9.7 | 0.6 |
| SPOC-18 | 72.1 | 49.1 | 1.0 |
| SPOC-19 | 22.2 | 21.3 | 0.6 |
| SPOC-20 | 64.3 | 43.3 | 2.0 |
| SPOC-21 | 38.3 | 22.1 | 3.5 |

* RED Patients fell outside White/Westbrook Agreement Boundaries

Since the Nasal Pressure is the major contributor to the model, we decided to evaluate the performance of a pleth only model (e.g. using data only from the pulse-oximeter). The best model parameters were:
SpO₂ Drop: discussed earlier
IR BE Energy: Breath effort signal as defined in the obstructive/central apnea section.
RED DC Drop Area: The area of the DC drop in the RED signal relative to a baseline. The baseline is as computed in the same way as in previous similar parameters.
Pleth Red AC HR Variability: the variability of heart rate measured in a 10 second window preceding the current breath.

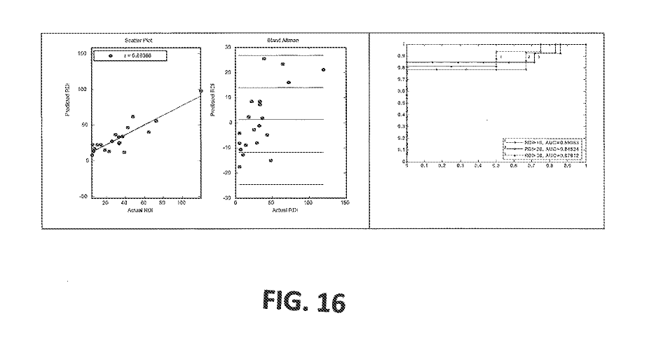

This model performed well, but not as well as the model that also included nasal pressure. FIG. 16 shows the correlation plot for RDI with a correlation coefficient of 0.894, with a bias of approximately 1 RDI point and precision of approximately 10. The ROC curves showed an AUC between 0.84 and 0.89 for the RDI>10, 20, 30 predictions.

For sensitivity analysis, events needed to be matched between the manual and predicted scoring. This matching then results in the labeling of events as true positive, false positive, and false negative (true negatives are ill-defined). The following rules (consistent with those used in De Almeida, et. al. “Nasal pressure recordings to detect obstructive sleep apnea”, Sleep Breath 2006 10(2):62-69) were applied for aligning and matching events:
The time at the center of each event, both manually scored and predicted, was used for alignment.
If a predicted event occurred within 10 seconds of an actual event, it was scored a true positive.
False negative events were those that were manually scored as an event without a predicted event within 10 seconds.
False positive events are when a predicted event was not within 10 seconds of a manually scored event.
If two predicted events occurred within 10 seconds of an actual event, one was scored a true positive, the other a false positive.

### White/Westbrook Diagnostic Agreement

As defined in “D. White, T Gibb, J Wall, P Westbrook, ‘Assessment of Accuracy and Analysis Time of a Novel Device to Monitor Sleep and Breathing in the Home’, Sleep, 18(2):115-126”, the diagnostic agreement rules are as follows:
Agreement defined as:
   AHI≧40 events per hour (e/hr) on both systems
   If AHI<40 on PSG, AHI within 10 e/hr on both
Overestimate of AHI defined as:
   AHI 10 e/hr greater on system than PSG (both <40 e/hr)
Underestimate of AHI defined as:
   AHI 10 e/hr less on system than PSG (both <40 e/hr)

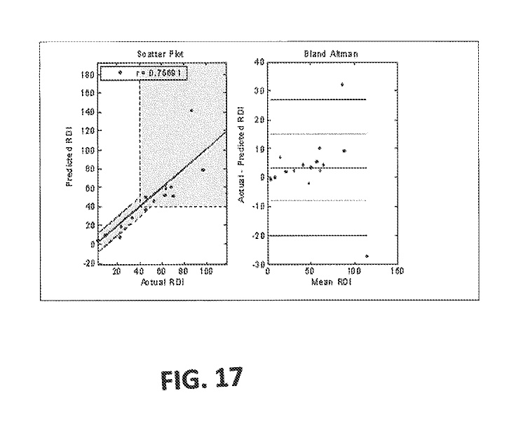

The most recent correlation plots show the diagnostic agreement regions with dashed lines. FIG. 17 shows the diagnostic agreement region in grey. In the example plot, only 1 of the data points falls outside the diagnostic agreement range.

### Kappa Agreement

Cohen's Kappa statistic provides the degree to which two judges concur in the respective classification of N items into k mutually exclusive categories—relative to that expected by chance. It is a “chance corrected proportional agreement”. Unweighted Kappa assumes no relationship between events, Linear weighted Kappa assumes numeric relationship (e.g. 1 is closer to 2 than it is to 3). An example epoch-by-epoch confusion matrix of a system prediction that has 90% agreement (always predicts zero events per epoch) is shown below. As expected, the Kappa value for this matrix is 0. To the right of the matrix is a set of generally accepted interpretations of the ranges of Kappa values.

System Prediction

| | | 0 | 1 | 2 | 3 |
|---|---|---|---|---|---|
| PSG | 0 | 8154 | 0 | 0 | 0 |
| | 1 | 870 | 0 | 0 | 0 |
| | 2 | 9 | 0 | 0 | 0 |

| kappa | Interpretation |
|---|---|
| <0 | No agreement |
| 0.0-0.19 | Poor agreement |
| 0.20-0.39 | Fair agreement |
| 0.40-0.59 | Moderate agreement |
| 0.60-0.79 | Substantial agreement |
| 0.80-1.00 | Almost perfect agreement |

Agreement Percent = 90.3%
Kappa = 0!

### Validation Set Results

The validation set consists of 15 patients. We ran an analysis of the SPOC data from this validation set and developed predictions of RDI and events. At this point, scoring information on the patients was utilized to fully analyze the results.

The patient population in the validation set was more severe than in the training set. The mean RDI for the training set was 33 with 20% of the patients having an RDI>40, while the mean RDI for the validation set was 53 with 60% of the patients having an RDI>40. The scored RDI and the predicted RDI for each patient are shown below.

| | RDI from Alice |
|---|---|
| | PSG Scoring |
| SPOC RDI | Report |
| 3.9 | 2.4 |
| 8.8 | 8.6 |
| 7.2 | 21.5 |
| 18.9 | 23.1 |
| 28.6 | 33.1 |
| 49.6 | 45.4 |
| 36.9 | 45.7 |
| 46.3 | 53.2 |
| 51.7 | 62.1 |
| 58.9 | 63.4 |
| 59.8 | 68.8 |
| 50.2 | 70.1 |
| 141.8 | 87.1 |
| 78.8 | 96.8 |
| 54.5 | 118.6 |

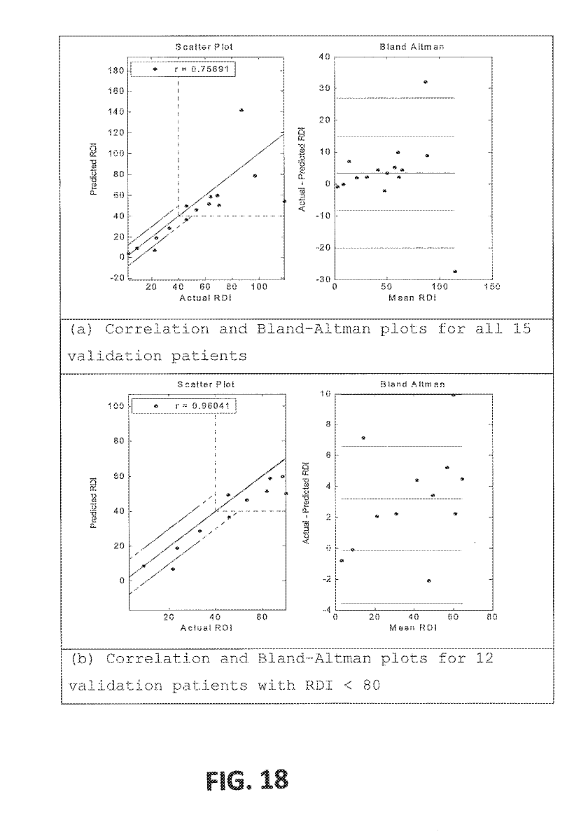

Although the population was somewhat different than the training set, the SPOC algorithms still performed quite well. The system correctly classified all severe (RDI>40) patients as severe. Although the RDI correlation is lower than in the training set, this was driven by two outliers with high RDI values (RDI>80). As shown in FIG. 18 the correlation coefficient for all 15 patients was 0.76 (bias=3, precision=10), while the correlation coefficient for patients with RDI<80 is 0.96 with a bias of 3 and precision of 3. The plots also show a diagnostic agreement of 93% missing only on SPOC-22 where the predicted value was 7 and the scored RDI was 20.

The table below shows the epoch-by-epoch analysis of the number of events. The Kappa statistic for the validation set was 0.47 which is slightly higher than the training set.

| | | System Number of Events | | | |
|---|---|---|---|---|---|
| | | 0 | 1 | 2 | 3 |
| PSG Sytem | 0 | 7064 | 1364 | 31 | 0 |
| Number of | 1 | 961 | 1969 | 18 | 1 |
| Events | 2 | 34 | 61 | 3 | 0 |

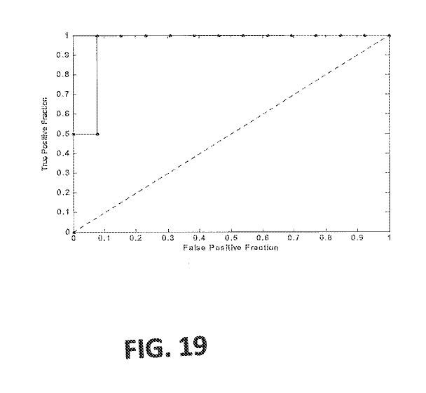

With only 2 patients in the validation set having an RDI<20 and both of them being less than 10, the ROC curves and AUC for RDI>10, 15, and 20 were all identical. The AUC was excellent at 0.96. The ROC for all three are shown in FIG. 19.

As discussed above with the AUCs for various RDIs, the AUC analysis with ODI in the validation set is of questionable validity due to the fact that only 2 patients have RDIs less than 20. The table of ODIs versus PSG RDIs is shown below.

| SPOC ODI | PSG RDI |
|---|---|
| 0.00 | 2.40 |
| 0.93 | 8.60 |
| 6.95 | 21.50 |
| 5.96 | 23.10 |
| 3.87 | 33.10 |
| 21.79 | 45.40 |
| 1.66 | 45.70 |
| 29.22 | 53.20 |
| 24.33 | 62.10 |
| 28.55 | 63.40 |
| 37.21 | 68.80 |
| 16.08 | 70.10 |
| 18.92 | 87.10 |
| 51.87 | 96.80 |
| 37.67 | 118.60 |

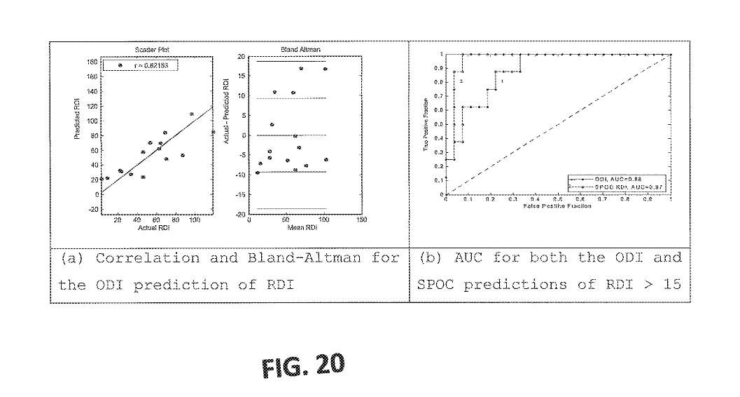

The correlation plot for ODI prediction of RDI (after linear scaling) are shown in FIG. 19. The correlation coefficient is only r=0.82 and the precision is 10 (after linear adjustment, the bias is 0 by definition). The ROC curves using both RDI and SPOC prediction for RDI>15 on all 35 patients (to get a better distribution of low RDI patients) is shown in FIG. 20. Notice that the SPOC RDI has an AUC of 0.97 whereas the ODI AUC is 0.88.

In the validation set, there were 3 patients we considered to be outliers: SPOC-22, SPOC-24, and SPOC-26 (although SPOC-24 and SPOC-26 were correctly classified as “severe”). The table of predicted versus manually scored RDIs in the validation set is shown below, with the outliers highlighted.

| | | Reported |
|---|---|---|
| | | SPOC |
| Patient | PSG RDI | RDI |
| SPOC-22 | 21.5 | 7.2 |
| SPOC-23 | 70.1 | 50.2 |
| SPOC-24 | 118.6 | 54.5 |
| SPOC-25 | 68.8 | 59.8 |
| SPOC-26 | 87.1 | 141.8 |
| SPOC-27 | 45.7 | 36.9 |
| SPOC-28 | 8.6 | 8.8 |
| SPOC-29 | 53.2 | 46.3 |
| SPOC-30 | 33.1 | 28.6 |
| SPOC-31 | 45.4 | 49.6 |
| SPOC-33 | 62.1 | 51.7 |
| SPOC-34 | 96.8 | 78.8 |
| SPOC-35 | 23.1 | 18.9 |
| SPOC-36 | 63.4 | 58.9 |
| SPOC-37 | 2.4 | 3.9 |

In our preliminary report of validation set results, we under predicted RDI for two of these (22 and 24) and over-predicted the RDI of SPOC-26. A closer look at SPOC-26 showed that there were four hours of time in which the pleth signal was “disconnected”. This type of error was not being detected by our algorithm at the time of testing. After correcting for this disconnection, however, the RDI estimate for SPOC-26 drops from 141 to 52 (although there were some disconnections in the other patients, none were long enough to significantly affect the scoring).

In analyzing the under-prediction that is prevalent for the high RDI patients, there appears to be two primary causes: (1) the SPOC system was trained on low and moderate patients in order to produce better diagnostic accuracy, and (2) there was a significant difference between sleep time and study time in a few patients.

In our models, a good example of how training on low and moderate patients affects the scoring of the severe patients is in calculating the baseline. Each parameter (such as DC Drop and SpO₂ Drop) calculates a “baseline” from which to compare the current breath. For patients with many events, this baseline is artificially more “severe” on average, which causes the current breath to seem less “severe” and allows a number of events to just miss their “threshold”. As described previously, in the Nasal Pressure Drop parameter we utilized two separate baseline calculations—one for moderate and mild patients and one for severe patients. With the increased number of severe patients in the validation set, it now appears that this methodology should be utilized more frequently in our models. Another approach is to create separate models for severe and non-severe patients (the SPOC system has proven its ability to determine the difference). Of course, an important consideration is whether fixing the RDI of severe patients is even an important issue if this device is to be used only for “screening”.

The second source of under prediction is the lack of accurate sleep scoring in the SPOC data. This issue is particularly relevant for SPOC-22 which is moderate and was our only diagnostic disagreement. The SPOC prediction of RDI was 7.2 whereas the PSG RDI was 21.5. However, patient 22 was awake for over half the night. During this waking period, the SPOC system predicted an RDI of close to zero causing the overall RDI to be artificially low. SPOC-22 was rather extreme in his wake time vs. sleep time, taking 86 minutes to fall asleep whereas the other patients averaged only 14 minutes to fall asleep. With a more appropriate estimate of sleep-time, the SPOC RDI prediction for patient 22 would have been 14, which would have been a diagnostic agreement. Improving sleep time estimates, if possible, would appear to be an effective means of improving the RDI prediction for mild and moderate patients.

The data driven approach has created a system that appears to be robust to differences in patient population and performs well relative to other systems on the market. The system uses a unique combination of nasal pressure, saturation, and plethysmography parameters and each of the 4 parameters contributes unique information that is utilized by the system. Although there were a few outliers in the validation set that produced a lower than expected correlation with RDI, these outliers are largely caused by two factors: (1) the difference between sleep time and valid data time (our surrogate for sleep), and (2) our focus on correctly discriminating mild and moderate patients. The largest outliers were limited to the very high RDI patients (RDI>80) and the RDI correlation for patients with RDI<80 was 0.96. Even with the sleep-time induced underestimates, the White/Westbrook diagnostic agreement was 93%. With compensation for this sleep time disparity, the diagnostic agreement was 100%.

### Example 4

In this study, 35 patients were examined from a sleep study in which a full array of polysomnography (PSG) parameters were collected alongside photoplethysmography (PPG) parameters collected by a single sensor on the alar site. The goal was to determine whether respiratory rate and IE ratio could be accurate determined using PPG alone.

In the 35 patients studied respiratory rate was reliably detected using PPG (r²=0.88). IE ratio, however, could not be determined through PPG alone, however. Simulations show that the process used to filter out the high frequency or cardiac component from PPG is responsible for removing IE ratio information from the signal. Because the cardiac component is by the far the strongest component of the signal, separating IE ratio from PPG may be impossible.

An algorithm to reliably remove respiratory rate from the IR and RED PPG signals has been developed. This algorithm processes the signal to effectively remove the cardiac component and DC shifts unrelated to respiratory effort.

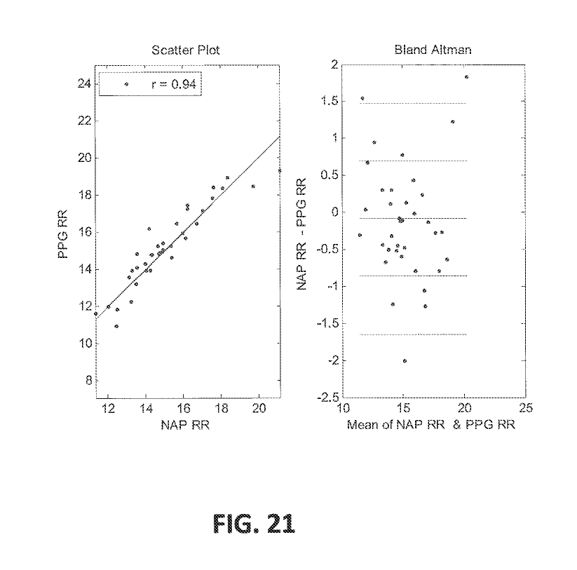

Over the course of a sleep study, this respiratory component effectively tracks the respiratory rate as determined by the nasal pressure. FIG. 21 shows how the PPG tracks the average respiratory rate of a sleeping patient.

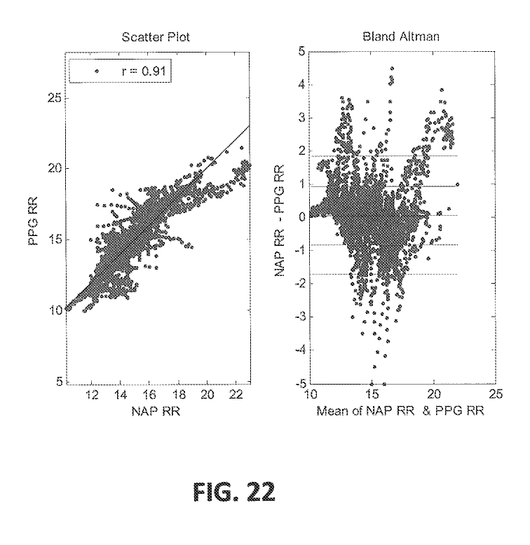

In addition to the long term average, a more short term respiratory rate was tested. FIG. 22 shows smaller one minute regions taken from the 35 patients. FIG. 22 shows 4,473 one minute regions of data. These regions were selected based on the following criteria:
1. Nasal pressure was not zero and was not saturated
2. PPG SaO2 was above 75%
3. No LED changes
4. IR and RED channels agreed on heart rate and respiratory rate

It should be noted here that even within these regions, nasal pressure is not 100% reliable and sections of noise exist in the NAP signal that the above criteria did not disqualify.

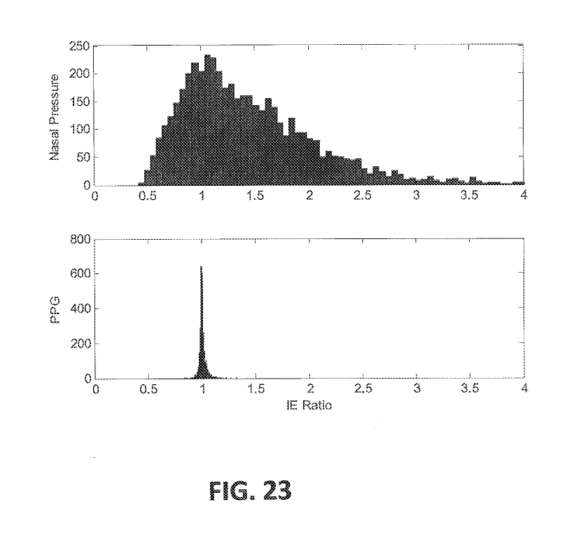

IE ratio as calculated by PPG did not correlate reliably with IE ratio calculated using NAP signal. The top panel of FIG. 23 shows a histogram of IE ratios calculated from one minute regions using the NAP signal. The bottom panel shows a histogram of IE ratios from the same regions calculated using the PPG signal. Whereas the NAP signal provides a wide spread of measured IE ratios, the IE ratios calculated from PPG are clustered around a 1:1.

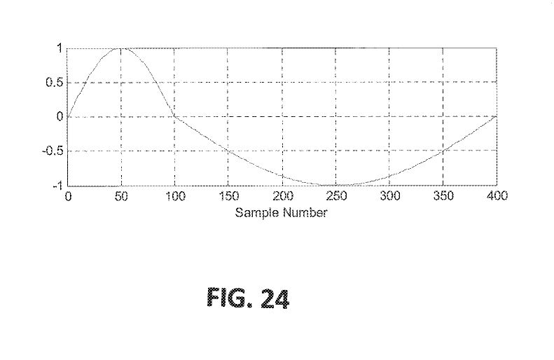

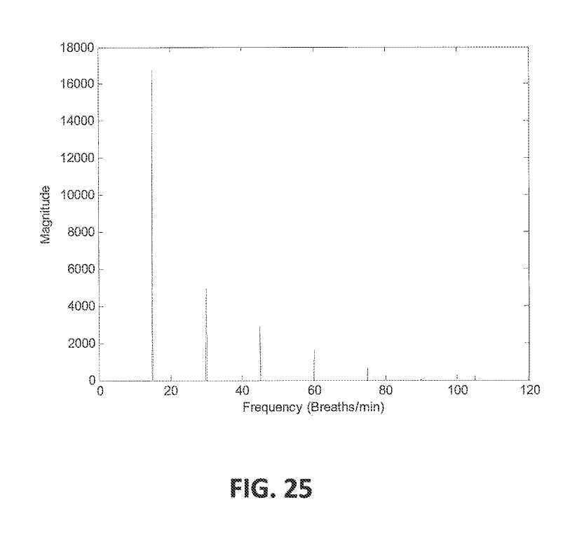

A simulation was conducted to investigate the reason for the absence of IE ratio information in the PPG signal. A test signal was generate with an IE ratio of 1:3 as shown in FIG. 24. FIG. 25 shows the frequency spectrum of this test breath.

Although the fundamental breath rate of this test signal is 15 breaths/min (0.25 Hz), the uneven IE ratios creates energy at harmonic frequencies (30, 45, 60, and 75 breaths/min). These higher harmonics enter the range of frequencies affected by the cardiac component. The same filtering algorithm applied to the sleep study to extract the respiratory component from PPG was applied to this test signal. The resulting signal is shown in FIG. 26. FIG. 26 shows that because the band pass filter for respiratory rate is tight to remove noise in adjacent frequency bands, the respiratory signal that remains is very close to sinusoidal (single frequency). This sinusoidal signal has very little I:E ratio information remaining. Some strategies were tested to better separate the I:E ratio from the PPG data in the sleep studies but thus far none have been successful. Other approaches exist, but this will require significantly more effort.

Conclusion:

The PPG is a reliable independent channel to determine respiratory rate and can therefore be a good compliment or backup to nasal pressure. The I:E ratio, however, is difficult to reliably extract from the PPG signal.

### Example 5

### Conscious Sedation

Provided below is an example of one procedure for administering conscious sedation to a patient using system and methods according to embodiments of the invention.

Prior to administration of CNS depressants or anesthetics to induce conscious sedation, monitors including, but not limited to, ECG and pulse oximetry (as part of PPG monitoring) are operatively attached to the subject. The patient is fitted with a “nasal pillow” system incorporating PPG and capnography to facilitate monitoring at the nasal septum, nasal alae, or both, to acquire combinations of the following parameters: oxygen saturation, respiratory rate, respiratory effort, capnography, venous capacitance and a surrogate for cerebral blood flow determined from the AC component of the PPG or the raw PPG signal obtained from a nasal alae or septum, with additional parameters derived from the PPG and other measurements optionally also being collected, analyzed and displayed, as discussed herein.

Once medication administration commences, a low level of CPAP sufficient to allow reliable end tidal carbon dioxide measurement is provided (in the range of 3-6 cm H₂O; adequate CPAP will be determined by analysis of the capnogram waveform), the system continuously monitors the subject for signs of respiratory depression, cardio-respiratory instability, or both, such that, should evidence of respiratory compromise be detected, the system automatically begins to titrate CPAP to maintain a patent airway and to improve oxygenation and gas exchange, with, optionally, alarms being set off to alert healthcare workers of early compromise and algorithms included in the system “advise” the proper action with prompts on the monitor screen.

If the addition of low levels of CPAP (<6 cm H₂O) corrects the respiratory compromise and the other monitored parameters remain stable, the procedure and administration of medications is permitted to continue. In addition, or alternatively, a narcotic reversal agent may be administered to the patient. If the addition of low level CPAP and/or narcotic reversal agency is inadequate to reverse the early signs of respiratory compromise, the system begins the administration of BiPAP or adaptive servo-ventilation. Simultaneously, healthcare workers receive further prompts on proper intervention and the system automatically reduces the infusion rate or shuts off the infusion pump, depending on the degree of respiratory compromise.

### Example 6

### PCA Infusion Pumps

The following protocol is provided as an example of embodiments of the invention wherein PCA pumps or other infusion devices are used to administer the opiod or other narcotic:

At the time of initiation of a PCA infusion, the patient is fitted with a “nasal pillow” system incorporating PPG and capnography to facilitate monitoring at the nasal septum, nasal alae, or both, to acquire combinations of the following parameters: oxygen saturation, respiratory rate, respiratory effort, capnography, venous capacitance and a surrogate for cerebral blood flow determined from the AC component of the PPG obtained from a nasal alae or septum, with additional parameters derived from the PPG and other measurements optionally also being collected, analyzed and displayed, as discussed herein above.

The nasal pillow system also incorporates or is operatively interfaced with an accelerometer or like motion sensing means for monitoring the level of activity of the subject, such that, as long as the subject is active, the system remains in a “surveillance” mode designed to markedly reduce the number of false alarms which lead to “alarm fatigue, but, when the patient is inactive, a “high alert” mode is initiated and the system monitors all parameters at a higher degree of scrutiny. The system continues to monitor the subject, continuously or at a pre-set intermittent rate, and at the earliest signs of respiratory distress (airway obstruction/increased effort, hypoxemia, hypercapnia) the system initiates CPAP.

If low pressure CPAP corrects the problem, the system continues to monitor the patient, but if low pressure CPAP is inadequate to reverse the early symptoms of respiratory depression/airway obstruction, a higher level of CPAP or BiPAP/adaptive servo-ventilator, is initiated, a narcotic reversal agent is administered, healthcare workers are alerted, and/or the rate of infusion on the PCA pump is reduced or the infusion is terminated

### Example 7

### SPOC Arrays with Oxygen Delivery

During delivery of CNS depressant to a subject, a first signal is acquired at a central source site of the subject and is monitored for evidence derivable from the first signal which is known to be indicative of hypoventilation. On detection of evidence of hypoventilation, a second signal is generated which is sent to a controller to (i) alert staff of the identified hypoventilation; (ii) to automatically initiate positive pressure ventilation of the subject; and, if the positive pressure ventilation does not produce evidence of resolution of hypoventilation in the subject, to (iii) decrease or stop delivery of the CNS depressant. In a particular embodiment implementing this exemplary application, a central controller extracts the information required from the central source site PPG signal to acquire the venous impedance signal from which evidence of increased breathing effort or decreased breathing rate or regularity is extracted. The controller, then, based on the evidence, and in a preferred embodiment, after confirming that no contradictory signal is being acquired from any other sensor, limits or turns off delivery of the CNS depressant unless/until the evidence of hypoventilation is resolved or trained personnel intervene.

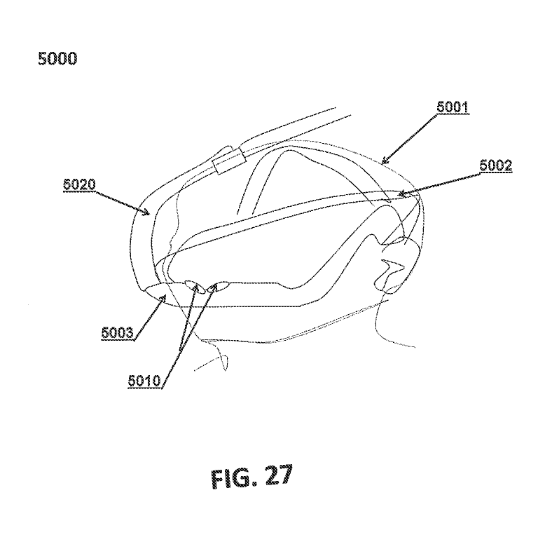

In FIG. 27, there is shown a system according to this invention, 5000, operatively adhered to a subject 5001, shown in outline. A harness system 5002 is shown for keeping an air exchange housing 5003 of a system 5000 in proper position and alignment on the face of the subject 5001. As will be seen from the further description below, the air exchange housing 5003 comprises means for sealingly measuring CO₂ in exhaled air, means 5010 for provision of positive pressure ventilation of the subject 5000, a source of gas, which is considered a fluid for purposes of this invention, 5020, which may include a source of high oxygen gas, ordinary breathing air, inhalational anesthetic or other volatile agents and the like. The source of gas 5020 is under control of the system of this invention, such that, upon detection of hypoventilation, the system initiates positive pressure ventilation, preferably with oxygen enriched air.

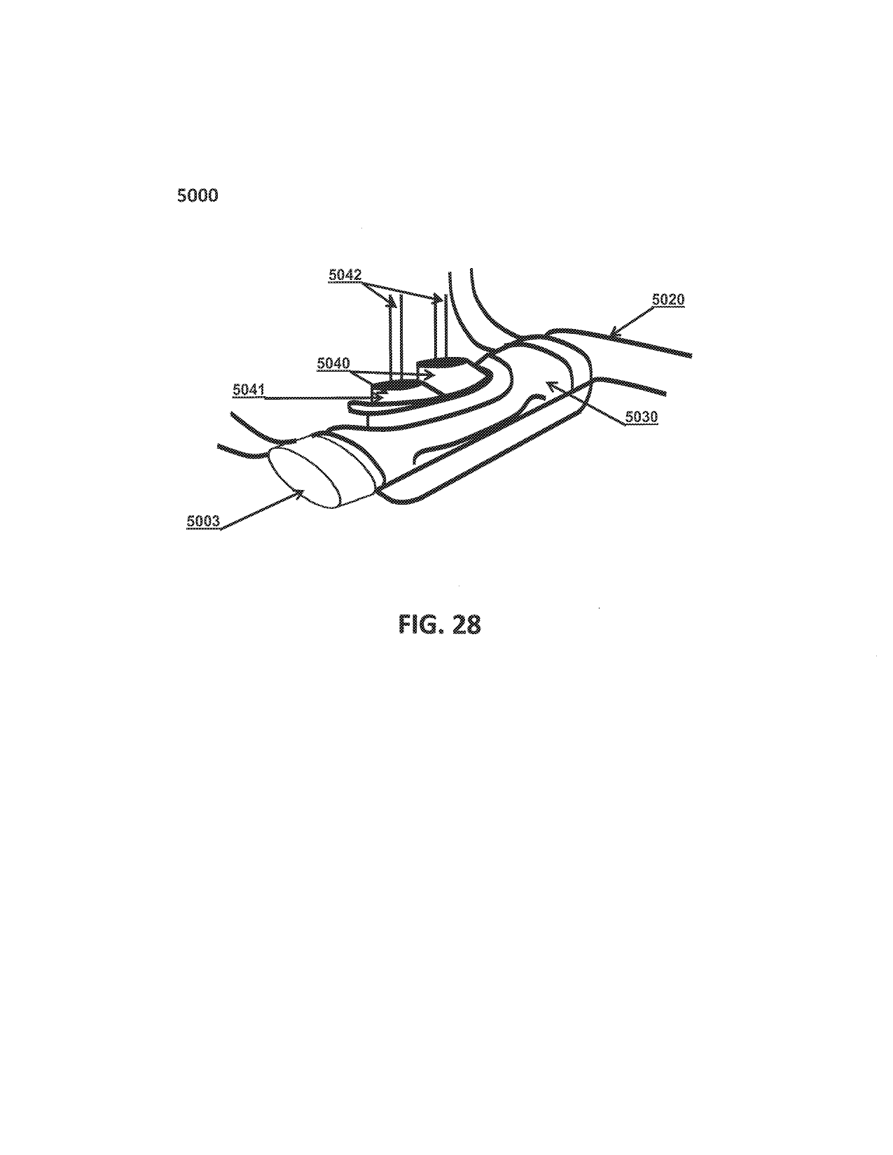

Referring now to FIG. 28, there is shown a detail of one representation of an Air Exchange Housing 5003 as shown in FIG. 27, with the source of gas 5020 connected to a housing unit 5030 into which positive pressure gas can be infused when/if the controller receives a signal indicating subject hypoventilation. For sealingly engaging with the nares of the subject, there are provided two “nasal pillows” 5040, each comprising a nasal seal 5041 running through which there is provided any number of tubes, channels or the like 5042 for provision of any or all of the elements of the various aspects of this invention, including but not limited to: means for measuring exhaled CO₂, e.g., a capnometer probe, electrical connections for a Central Source Site PPG probe, (i.e., both for at least one photodiode or the like and at least one photodetector, or the like, for which wavelengths of illumination and detection may be multiplexed, according to methods known in the art), to acquire PPG signals, pulse oximetry signals or both, means for delivery of pharmacologic agent(s) or fluids to the nasal septum.

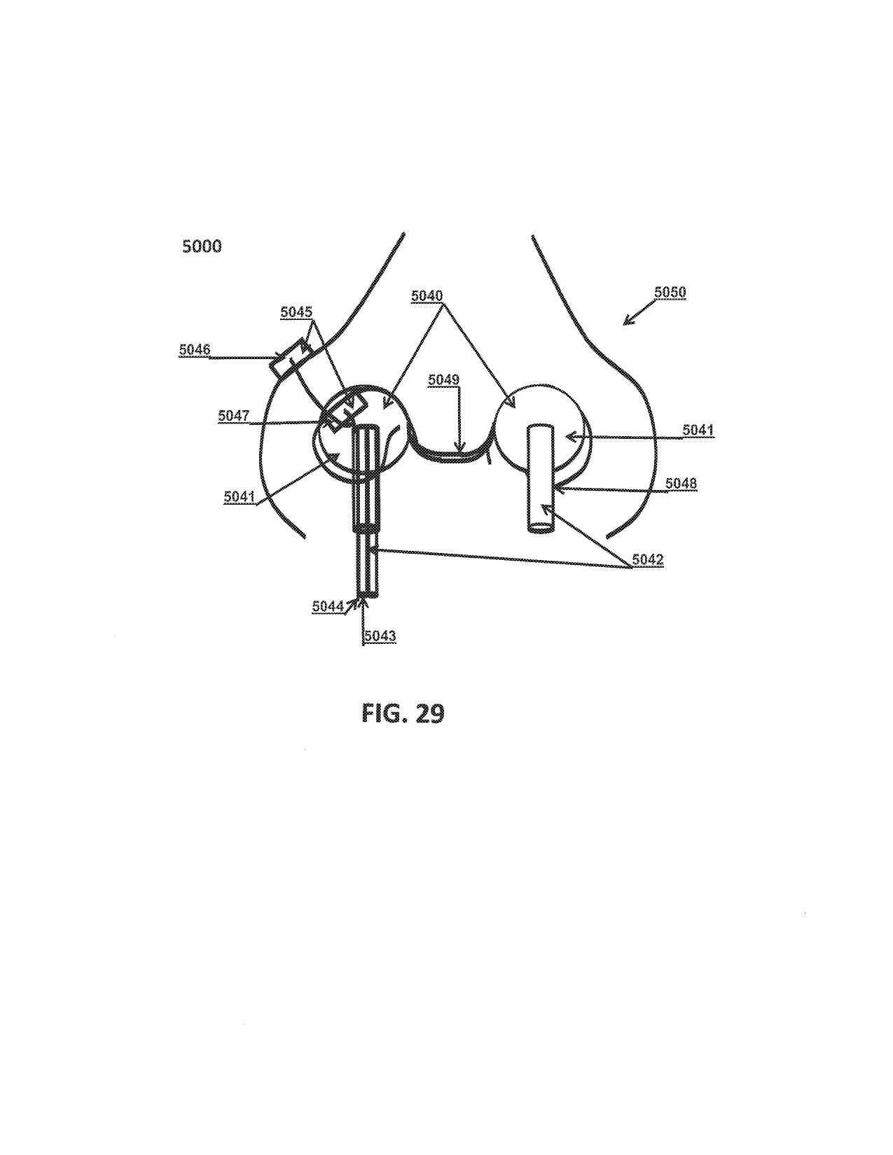

In FIG. 29, there is provided a detailed, from below view, of one embodiment according to this invention of a nasal interface of the nasal interface unit 5050 which provides a representation of various elements of a system 5000, method and appratus, from this rather unique angle of the human anatomy. Looking upward into the nares of a subject, there is shown two “nasal pillows” 5040, each comprising a nasal seal 5041 running through which there is provided any number of tubes, channels or the like 5042 for provision of any or all of the elements of the various aspects of this invention, including but not limited to: means for measuring exhaled CO₂, e.g. a capnometer probe 5043, electrical connections for a Central Source Site PPG probe 5044, (i.e., both for at least one photodiode 5046 or the like and at least one photodetector 5047, or the like, for which wavelengths of illumination and detection may be multiplexed, according to methods known in the art), to acquire photoplethysmographic signals, pulse oximetry signals or both, and/or means 5048 for delivery of pharmacologic agent(s) or fluids to the nasal septum, as described elsewhere in this application. The assembly of different elements described in this example may be such that each element with respect to each other element is held in good registration with the physiology of the subject by an alignment member, 5049, for example, which registers the assembly to the nasal septum. Each of the elements may be likewise held in pliant registration with each other element of the system and in relation to the alignment member 5049. Referring back to other figures, examples and disclosure provided herein, one skilled in the art will appreciate how an infusion apparatus may be controlled by acquisition of PPG signal from a central source site to measure subject physiologic parameters, and to control, on the basis of analysis of the central source site PPG signal, infusion of anesthetic, other pharmacologically active agents and/or fluids.

### Example 6

### Narcotic-Reversal Administration

A software-based system can provide the decision making capability to operate syringe pumps, which have been available for many years. In a preferred embodiment, all these devices can be combined in one device (or linked by communication protocols known in the art) to provide a safer alternative for these patients. In one embodiment, the system operates in conjunction with a PCA pump apparatus. In an alternate embodiment, the system replaces the obsolete PCA pump apparatus.

According to the present invention, the system, method and apparatus includes an end-tidal CO₂ monitor sampling exhaled CO₂ next to the nose through a small tube alongside the nasal cannula delivering oxygen inside the nose, with the sampled exhaled CO₂ generating a wave form and respiratory rate that is displayed, recorded and sent to a computer or equivalent structure programmed to detect alarm conditions that sends a signal to one or more existing syringe pumps that respond by injecting the life saving naloxone or other drug-reversal agent in the patient's intravenous line. In a particular embodiment of the invention, a photoplethysmography signal is acquired from the patient at a central source site such as the nasal alar and the signal is processed to reveal respiratory rate, respiratory effort or both. As a further safety feature of the present invention, the administration of narcotic-reversal agent is made dependent on concurrent acquisition of end-tidal capnography information and PPG signals.

According to this embodiment of the invention, when a decision is made to administer naloxone, in a preferred embodiment, it is simultaneously delivered through an oxygen-supplying nasal cannula tube with a disposed aerosol nozzle or a separate aerosol delivery system, as a nasal spray to be absorbed, either as the sole method of supplying the antidote, or as a fail-safe backup mode in the event the intravenous line does not exist or is faulty, or there is a failure in the PCA pump, either human or design.

These components could further be connected and made to function with the well-known RS-232 interface, for example.

There are several commonly used drugs in resuscitation scenarios, and much time and effort could be saved by having such drugs pre-packaged, so that a staff member could simply press one button, and the device, which is already plugged into the patient's IV, could deliver the intended resuscitation drug. Possible drugs include but are not limited to naloxone (reverse narcotic), D 50 (sugar to reverse insulin overdose), sodium bicarbonate (to reverse high potassium and acidosis), Romazicon/flumazenil (to reverse benzodiazepines), glycopyrrolate (Robinul) or atropine (to speed up a slow heart), phenylephrine to safely increase blood pressure without speeding up the heart), epinephrine/adrenalin to raise the blood pressure and speed up the heart, facilitate defibrillation, treat shock and severe allergic reaction and shock). Esmolol (safe short acting drug to slow down the heart), Vasopressin (drug for severe “vasodilatory” shock), and Cardizem and Adenosine to slow rapid heart rhythms.

In the event of a failed or unobtainable intravenous access, the device could also permit some or all of the drugs to be delivered intra-nasally. For example, in various embodiments, naloxone and other drugs are provided through a nasal cannula designed with an aerosol delivery system, either in addition to or in lieu of intravenous delivery. As it is known that naloxone presents little to no risk of adverse effects or overdose, a particular embodiment contemplates administering naloxone or similar agents intravenously or intranasally.

According to a particular embodiment, naloxone is pre-loaded in a tamper-proof cassette or syringe-injector. For example, proprietary naloxone loads may be used with the injector to avoid it being used for any other purpose (naloxone is harmless if injected rapidly, and other medications could be harmful if delivered fast in a norm-proprietary user-accessible device). The injector could deliver intravenously and/or intra-nasally through a nasal oxygen cannula plugged into the Apnea Rescue-Bot in response to Apnea Condition.

The device could provide further an assessment of pain control based on respiratory rate or quality of end-tidal CO₂ tracing, and advise whether the patient could safely tolerate more narcotic without respiratory depression, thus improving both comfort and the safety of patients. For example, respiratory rates greater than 20 breaths per minute with a high quality capnograph tracing may allow an increase in narcotic dosing, particularly with confirmation from the PPG signal acquisition that the patient is not experiencing respiratory rate depression or increased respiratory effort. The patient also be allowed more frequent opportunities to self-medicate safely, without demanding more of nursing personnel. Voice-activated patient requests could be evaluated and decided upon by the device if respiratory parameters were reasonable and no alarm conditions were being approached. According to this embodiment, all actions, alarms, and adjustments would be recorded, displayed, automatically entered into the EMR (Electronic Medical Record) or wirelessly relayed to the nursing station if desired.

In another embodiment of the device, other therapeutic medications besides the narcotic reversal agent could be given intravenously or intra-nasally. For example, phenylephrine, used commonly as a vaso-constrictor to relieve nasal congestion, is well known to have the side effect of elevating blood pressure. This side effect could be exploited as a remedy for dangerously low, blood pressure with nasal administration of the antidote, at least until an intravenous line could be established for the best support of low blood pressure. In addition, dangerously slow heart rates could be safely raised with dosages of glycopyrollate or atropine, dangerously fast heart rates could be slowed with Esmolol (which is metabolized in several minutes), and dangerously high blood pressure could be lowered with any number of medications in judicious amounts. Thus, the invention has another embodiment as a “Critical Care Rescue-Bot,” which may supply the necessary dosages either intravenously or intra-nasally in the event of intravenous line failure or prior to establishing an intravenous line, which occurs commonly).

According to varying embodiments described herein, the system and apparatus described above may all be controlled by a control system, such as a programmable logic controller or relay-based control system, with accompanying algorithms to govern the relationship between the monitoring inputs, the events or conditions and subsequent reporting or alarming for notification to hospital staff or other caregivers, as well as the actual automation of the various drugs being supplied to the patient. Such control systems that are now known or developed in the future are contemplated with and considered within the scope of the present disclosure.

It is to be expressly understood that uses for capnography monitoring devices as well as PPG monitoring devices, other than the uses described above, are contemplated for use with the apparatus and method of the present disclosure. The device could easily be used in home health scenarios, for example. As described above, there could be a very basic device for patients with sleep apnea. Currently, patients use CPAP machines (continuous positive airway pressure machines) with tightly fitting masks to force oxygen through obstructed and collapsed airways, and it would be advantageous to have a monitoring capability on these machines that could stimulate, them audibly or electrically. In various embodiments, naloxone delivery on confirmation with PPG acquired and processed signals may be provided through known CPAP machines.

In various embodiments of the present invention, home health systems and features are provided. For example, patients who may generally qualify for discharge from a primary care facility (e.g. hospital), yet ma still be at risk for over-sedation with prescribed narcotic-opiate pain pills, and chronic pain or cancer patients requiring administration of narcotics could be monitored and/or treated in situations outside of a hospital or primary care facility with various embodiments of the present invention.

For example, it is contemplated that a scaled-down version of the invention may be provided wherein an oxygen source comprises a portable oxygen tank rather than a wall-source, and various additional system components as shown and described herein are provided in sizes and formats adapted for home use. In particular, in the home environment, the patient is unlikely to be intubated, in which case end tidal CO₂ monitoring may become unreliable as an indicator of respiratory depression. In that scenario, the primary indicatory of the need to limit or stop narcotic administration, initiated positive pressure ventilation and, in extremis, administer narcotic reversal agent such as naloxone, is driven by signals acquired by PPG. As disclosed herein, in at least one embodiment, narcotic administration is limited or stopped when the PPG signal indicates respiratory depression, via, for example, the pump-agnostic delivery tube restrictor disclosed herein and in PCT/US11/46943, by means of which the line between the pump and the patient carrying narcotic is constricted or completely blocked.

in various embodiments, a system is provided that includes the ability to meter, monitor, and/or detect the amount of a narcotic dispensed to a patient in one embodiment, data related to the amount of a narcotic or pain-relieving drug provided to a patient (e.g. through a PCA pump) is continuously monitored and automatically compared with relevant patient information such as age, weight, gender, etc. Relevant patient information may be manually input into the system, such as through manual data entry at a terminal or interface upon check-in or admittance to a hospital.

Alternatively, relevant patient information may be automatically obtained from pre-existing medical records. In one embodiment, a system is provided with predetermined limits for various types of dispensed drugs and related patient information. In this embodiment, when the predetermined limits are exceeded, dispensing of drugs is at least temporarily prevented and/or naloxone or other reversal agents are dispensed to the patient.

While various embodiments of the present disclosure have been described in detail, it is apparent that modifications and alterations of those embodiments will occur to those skilled in the art. However, it is to be expressly understood that such modifications and alterations are within the scope and spirit of the present disclosure; as set forth in the following claims.

### Example 7

### Trauma Environment Treatment (TET) Ensemble

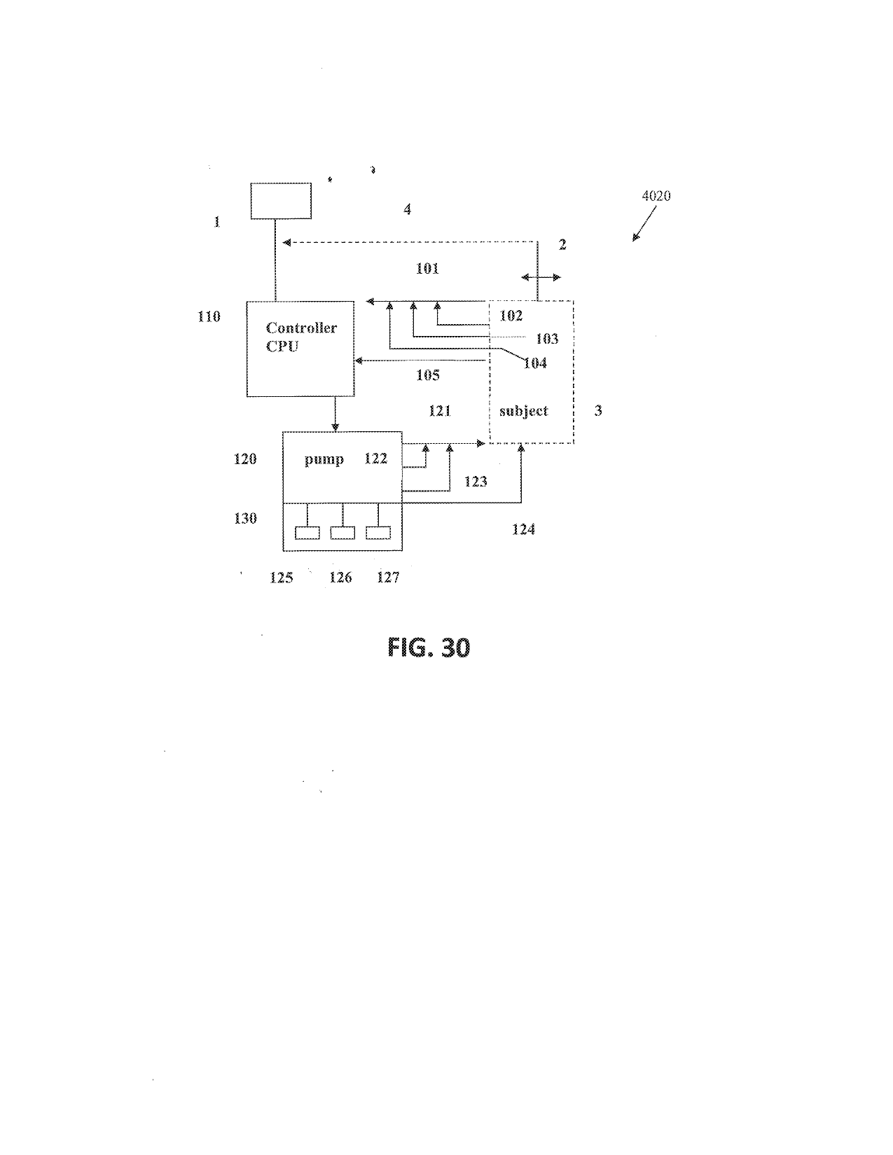

In particular embodiments, the TET system may include some or all of the following elements. Numerals in the following description reference a figure (first numeral) followed by a second numeral for a given element, separated by a slash. Thus, 1/1 references element 1 in FIG. 30, 30/3 references element 3 in FIG. 30, etc.

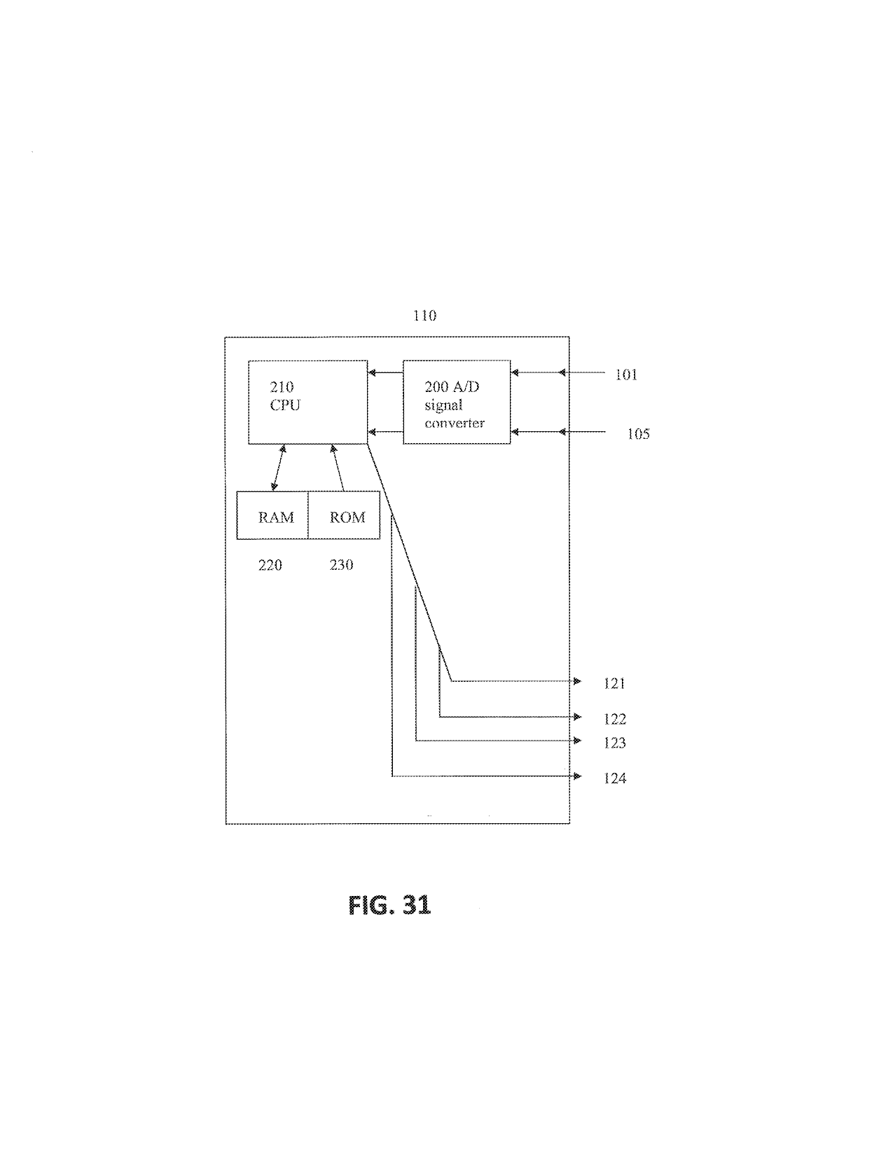

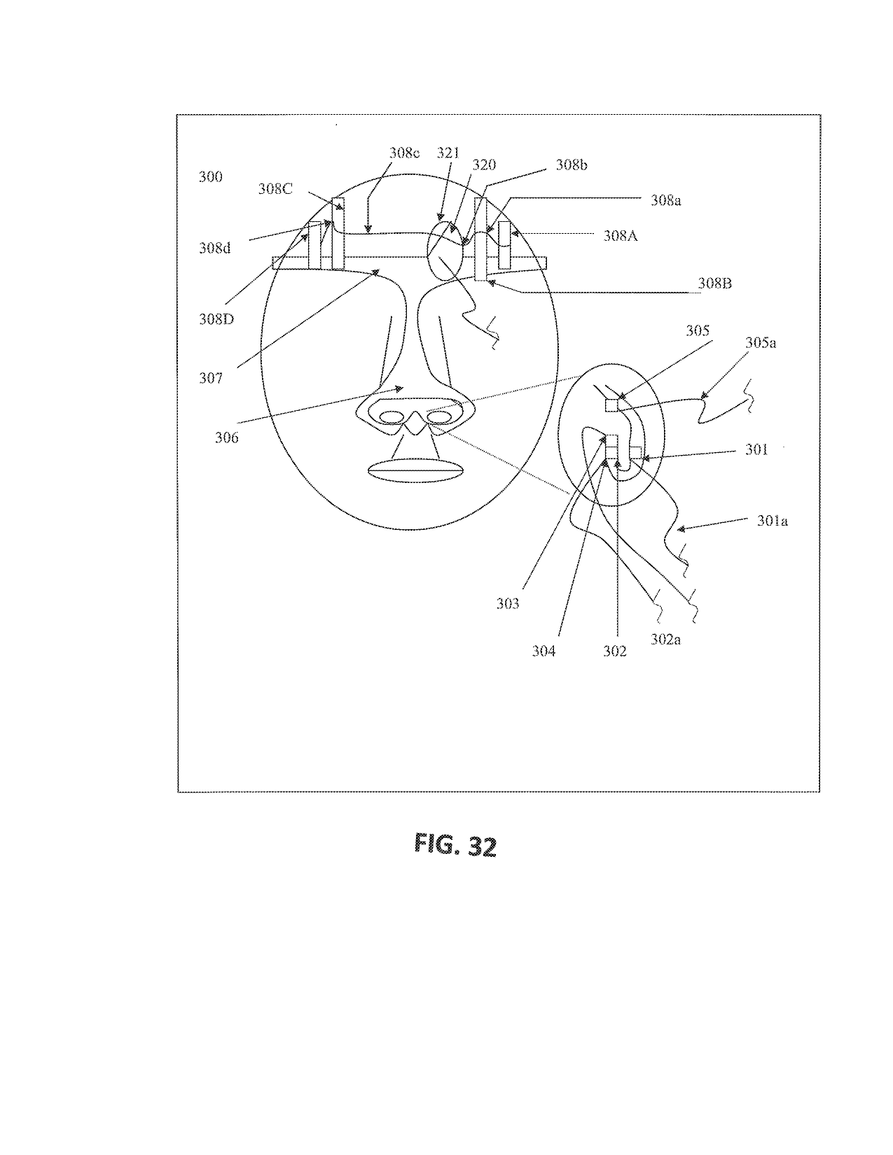

1. A battery pack or access to existing power in the TET ensemble 30/1.
2. An accelerometer or other motion (tilt, orientation, motion, elevation, or the like) sensing device 30/2 worn on the helmet of a subject 30/3 or other location on the head (e.g. behind the subject's ear) provides signals indicating whether a subject is actively moving or is inactive. This component is used primarily to “wake-up” the sensing system 30/4 so that it may remain in a standby status until needed. This reduces power consumption and the incidence of “false alarms”. The accelerometer signal is a separate signal from PD and/or PK signals acquired by sensors for reading such parameters from the subject. Further, lack of movement by the subject especially in a recumbent (supine or prone) position may be indicative of a serious injury. The data from the accelerometer in conjunction with data from SPOC can be used assess whether a subject is injured or if the activity detected is very regular and vigorous, this may be indicative of seizure activity, as from a concussive head injury from an IED. Once wakened, the controller comprising a CPU 30/110 receives data 30/102, 30/103, 30/104, 30/105 from the sensing device adhered to the subject 30/3, and, based on that acquired information, the controller/CPU 30/110, initiates delivery via a pump 30/120 of fluids and/or pharmacologically active agents 30/125, 30/126, 30/127, maintained in a secure compartment 30/130. These agents 30/125-30/127, for example, including but not limited to agents for providing analgesia, fluids and the like, are then infused via lines 30/122, 30/123, 30/124, optionally via a common line 30/121. As shown in FIG. 31, the outputs via lines 31/101 and/or 31/105 are received by an analog to digital converter if necessary 31/200 which transmits the signals to the CPU 31/210, which has stored in RAM 31/220 and/or ROM 31/230 appropriate signal processing algorithms for interpretation of the incoming subject physiologic information 31/101, 31/105, for outputting instructions to initiate infusion to the subject of appropriate fluids and/or pharmacologically active agents, 31/121, 31/122, 31/123, 31/124.
3. As shown in FIG. 32, at least one, and preferably two SPOC sensor assemblies 32/300 each containing pulse oximeter components (LED 32/301 and photodiode 32/302), nasal pressure sensors, 32/304, and in one embodiment, one of two ECG electrodes, 32/305 (the other to be placed in the undergarments or on the torso of the subject). Such components are known in the art, for example, for obstructive sleep apnea (OSA) monitoring. As shown in FIG. 32, one SPOC sensor assembly, 32/300, is affixed to each nasal ala and joins below the bridge of the nose to form a single device that can be easily emplaced by the subject or treatment provider. In alternate embodiments, SPOC units consist of a unit that is attached to single alae. However, the redundancy, improved fixation and additional access to the nasal epithelium makes a dual SPOC a preferred embodiment according to this aspect of the invention.
4. Means are provided to fix the SPOC sensors securely to the subject. For example, the sensor assembly may be affixed by a retainer device, 32/306, which fits over the bridge of the subject's nose and/or up to the helmet or other fixation point on the forehead, for example, using a headband, 32/307. The forehead band, 32/307, communications ensemble or the helmet optionally contain reservoirs of medications and or fluids, 32/308 (32/308A, 32/308B, 32/308C, 32/308D represent separate reservoirs with same or different fluids/medications), each of which is linked (via communication lines 32/308a, 32/308b, 32/308c, 32/308d to and activated for release of fluid/medications by the computer/CPU 32/320 which controls the closed-loop system, and other components/sensors of the system. The computer/CPU, 32/320, receives signals, 32/321, from the PD, PK or PD+PK sensors 32/301, 32/302, 32/305, affixed to the subject via communication line(s) 32/301a, 32/302a, 32/305a.
5. In some embodiments, as shown in FIG. 32, a small tube, 32/303, is incorporated into the assembly and is placed inside the subject's nostril and is pointed toward the nasal septum (nasal epithelium/mucosa, such as Kiesselbach's plexus and/or to the nasal epithelium/mucosa of the nasal turbinates) and delivers aerosols or non-aerosolized fluids, preferably in pre-metered doses of medications (e.g., opioids, anxiolytics, steroids, vasoactive drugs, and the like) using appropriate fluid delivery systems known in the art which are adapted for particular target delivery modes as described herein. Thus, for an intranasal delivery site, e.g., for delivery to the nasal epithelium, as shown in the drawings, a fluid nozzle aimed at the nasal mucosa is incorporated into a nasal alar attachment housing. For intravenous delivery, a tube with an IV needle, such as those known in the art, may be used. Based on the present disclosure, those skilled in the art may develop any number of equivalent delivery means to those described herein for delivery to any appropriate subject. Thus, in alternate configurations, the delivery device may be a needle or catheter which is to be inserted intravenously, intraperitoneally, intraosseously, intracardiacly, or the like, but the non-invasive assembly for intranasal delivery is shown in this embodiment.
6. Where utilized, the intranasal tube, 32/303, is connected to a drug delivery system capable of providing medication through the nasal epithelium delivery tube using aerosolized and/or non-aerosolized-based systems 32/303. The aerosolized and/or non-aerosolized medication(s) is/are optionally stored in pressurized canisters, 32/308, adapted to provide metered doses upon actuation of a valve or a small pump that delivers aerosolized and/or non-aerosolized doses from a given container, 32/308, via delivery line(s) 32/309 connected to said nasal epithelium delivery tube 32/303. The components of this device should be tamper-proof to prevent use of stored medications for other than intended purposes. Alternatively, the canisters 32/308 may be housed elsewhere on the subject, such as on a belt, which may also house the computer/CPU 32/320, pump if required 32/321 and communication lines and fluid delivery lines (32/308a-d and 32/309, respectively). The medication canisters or backup or replenishment containers are optionally carried independent of the other components of the system by a limited number of individuals responsible for the canisters and made available to personnel in need of the given medications. Medications in the canisters are optimized to maintain pharmacological potency under a wide range of temperature and atmospheric conditions, for example, by inclusion in the medication compositions appropriate preservatives and the like. Using parameters obtained from the SPOC array, medications can be metered to optimize delivery to the nasal mucosa.
7. Optionally, nitric oxide, histamine, methacholine or the like is included in the medication delivery system, either as part of the medication compositions or as a separate feed to the nasal mucosa, to increase permeability of the nasal mucosa to the delivered medications.
8. Highly concentrated doses of opioids (fentanyl, sufentanyl, and the like); opioid antagonists (naltrexone/naloxone for “recovery” if too large a dose of opioids is delivered); vasoactive drugs, particularly vasopressin; steroids (dexamethasone and others); dissociative agents such as ketamine; anxiolytics (benzodiazepines, gabapentin, pregabalin) and the like, are included as single component compositions which are separately deliverable to a subject in need of such agents, based on measurements of their PD parameters. Such medications are provided via separate infusion lines to the subject or may be combined for delivery through a single line. In this regard, reference is made to WO2011/149570, the disclosure of which is incorporated herein by reference.
9. Canisters or containers for medications and fluids, 32/308, are adapted so that they can be removably but securely inserted into the system (e.g., canisters or container that can be snapped into the system by engaging clips and holding compartments adapted for protection and engagement of such canisters or containers) so that different medication combinations can be provided. At least two drug or drug combinations are separately deliverable in an embodiment utilizing two SPOC sensors (one on each nasal alar).
10. A small central processing unit (CPU), 31/210, 32/320, including algorithms/software stored in RAM, 31/220, and/or ROM, 31/230 facilitate closed-loop (servo) delivery of medications and control of the medical devices (sensors and infusion mechanics).
11. Small infusion pumps (e.g., ambIT PCA pump), 32/321, deliver volume expanders (hypertonic saline; dextrans) via subcutaneous, intraosseous, or IV routes when available. This also extends the range of the TET to other levels (II-V) of medical care.
12. A second “peripheral” pulse oximeter sensor (fingers, toes, ear, etc) to provide information on volume status, or the status of an injured extremity. This is a standard finger/toe pulse oximeter probe/sensor which can be clipped (usually with a spring loaded design) to a finger or toe. The sensor usually contains two LED photodiodes (one emitting light in the IR range and one emitting red light). A photodetector evaluates the IR and red signals as well as the background signal sequentially and the pulse oximeter calculates the SpO₂ by calculations well known in the art. In the present application the sensor may be connected directly by a cable, or more advantageously by a Bluetooth or other wireless connection to the computer. The ability to simultaneously measure SpO₂ and PPG from two sites allows evaluation of volume status and/or status of a compromised extremity. See for instance U.S. Pat. No. 6,909,912.
13. Nasal pressure and/or flow sensors, 32/304, and/or PPG sensors, 32/301, 32/302, are utilized to detect phase of respiration and meter doses of medication only during the inspiratory phase.

Though the present disclosure has included description of one or more embodiments and certain variations and modifications, other variations and modifications are within the scope of the disclosure, e.g., the use of a certain component described above alone or m conjunction with other components may comprise a system, while in other aspects the system may be the combination of all of the components described herein, and in different order than that employed for the purpose of communicating the novel aspects of the present disclosure. Other variations and modifications may be within the skill and knowledge of those in the art after understanding the present disclosure.

We claim:

1. A method of monitoring and treating respiratory depression comprising:
securing a photoplethysmography (PPG) sensor to a central source site of an individual;
administering a central nervous system (CNS) depressant to the individual;
processing PPG signals from the PPG sensor with a controller in communication with the PPG sensor; and
administering a narcotic reversal agent to the individual if the PPG signals or a physiological parameter derived therefrom are outside a preset value range.

2. The method of claim 1, wherein the narcotic reversal agent is administered to the individual if a respiration rate of the individual is outside the preset value range.

3. The method of claim 1, wherein the narcotic reversal agent is administered to the individual if a respiratory effort of the individual is outside the preset value range.

4. The method of claim 1, wherein the narcotic reversal agent is naloxone.

5. The method of claim 1, further comprising securing to the individual an additional sensor configured to determine at least one parameter selected from respiration rate, end-tidal carbon dioxide content, blood pressure, heart rate and heart rate variability.

6. The method of claim 5, wherein the narcotic reversal agent is administered if (a) the PPG signals or a physiological parameter derived therefrom are outside a first preset value range; and (b) a parameter determined by the additional sensor is outside a second preset value range.

7. The method of claim 1, further comprising measuring a concentration of a component in the individual's breath.

8. The method of claim 7, wherein the component in the individual's breath comprises the CNS depressant and/or a metabolite of the CNS depressant.

9. The method of claim 7, further comprising securing to the individual an apparatus configured to supply oxygen.

10. The method of claim 9, further comprising administering oxygen to the individual if the PPG signals or a physiological parameter derived therefrom are outside the preset value range.

11. The method of claim 9, wherein the apparatus for supplying oxygen administers oxygen to the individual automatically when the PPG signals or a physiological parameters derived therefrom are outside the preset value range.

12. The method of claim 1, wherein the CNS depressant is administered by a device selected from the group consisting of a patient-controlled analgesia pump, an automatically administered closed loop infusion pump and an open loop intravenous infusion pump.

13. The method of claim 1, wherein the controller directs the device administering the CNS depressant to decrease the supply of the CNS depressant to the individual if the PPG signals or a physiological parameter derived therefrom are outside the preset value range.

14. The method of claim 1, further comprising impinging a feed line of the CNS depressant-administering device if the PPG signals or a physiological parameter derived therefrom are outside the preset value range.

15. The method of claim 14, wherein the controller automatically directs an occluding device to impinge the feed line when the PPG signals or a physiological parameter derived therefrom are outside the preset value range.

16. The method of claim 1, wherein the central source site of the individual is the nasal septum or the nasal alar.

17. The method of claim 1, further comprising alerting medical personnel if the PPG signals or a physiological parameter derived therefrom are outside the preset value range.

18. The method of claim 1, further comprising alerting the individual if the PPG signals or a physiological parameter derived therefrom are outside the preset value range.

19. The method of claim 18, wherein alerting the individual comprises directing an alerting device to provide a wisp of air to the face of the individual if the PPG signals or a physiological parameter derived therefrom are outside the preset value range.

20. The method of claim 1, wherein the controller is in wireless communication with the PPG sensor.

21. The method of claim 1, wherein the controller is in wireless communication with the device that administers the narcotic reversal agent.

22. The method of claim 1, wherein the CNS depressant is an analgesic agent.

23. A system for monitoring and treating respiratory depression comprising:
a PPG sensor configured to secure to a central source site of an individual;
a device configured to administer a narcotic reversal agent to the individual; and
a controller configured (1) to receive and process PPG signals from the PPG sensor, and (2) to direct the device to administer the narcotic reversal agent to the individual if the PPG signals or a physiological parameter derived therefrom are outside a preset value range.

24. The system of claim 23, wherein the controller is configured to direct the device to administer the narcotic reversal agent if a respiratory rate of the individual is outside the preset value range.

25. The system of claim 23, wherein the controller is configured to direct the device to administer the narcotic reversal agent if the respiratory effort of the individual is outside the preset value range.

26. The system of claim 23, further comprising an additional sensor that is configured to secure to the individual, whereby the controller is configured to receive signals from the additional sensor to determine at least one parameter selected from respiration rate, end-tidal carbon dioxide content, blood pressure, heart rate and heart rate variability.

27. The system of claim 26, wherein the controller is configured to direct the device to administer the narcotic reversal agent if (a) the PPG signals or a physiological parameter derived therefrom are outside a first preset value range; and (b) a parameter determined from signals generated by the additional sensor is outside a second preset value range.

28. The system of claim 23, further comprising an additional sensor configured to determine the concentration of a component in the individual's breath.

29. The system of claim 23, wherein the component in the individual's breath comprises the CNS depressant and/or a metabolite of the CNS depressant.

30. The system of claim 23, further comprising an apparatus configured to supply oxygen to the individual.

31. The system of claim 30, wherein the controller is configured to direct the apparatus configured to supply oxygen to increase the supply of oxygen to the individual if the PPG signals or a physiological parameter derived therefrom are outside the preset value range.

32. The system of claim 23, wherein the controller is further configured to alert medical personnel if the PPG signals or a physiological parameter derived therefrom are outside the preset value range.

33. The system of claim 23, further comprising a device configured to administer a CNS depressant to the individual.

34. The system of claim 33, wherein the device configured to administer the CNS depressant is selected from the group consisting of a patient-controlled analgesia pump, an automatically administered closed loop infusion pump and an open loop intravenous infusion pump.

35. The system of claim 34, wherein the controller is further configured to direct the device configured to administer the CNS depressant to decrease administration of the CNS depressant to the individual if the PPG signals or a physiological parameter derived therefrom are outside the preset value range.

36. The system of claim 33, further comprising an occluding device, wherein the device configured to administer the CNS depressant comprises a feed line and the controller is further configured to direct the occluding device to impinge the feed line if the PPG signals or a physiological parameter derived therefrom are outside the preset value range.

37. The system of claim 23, wherein the central source site of the individual is the nasal septum or the nasal alar.

38. The system of claim 23, further comprising an alerting device, wherein the controller is further configured to direct the alerting device to alert the individual if the PPG signals or a physiological parameter derived therefrom are outside the preset value range.

39. The system of claim 38, wherein the alerting device is configured to provide an auditory alarm if the PPG signals or a physiological parameter derived therefrom are outside the preset value range.

40. The system of claim 39, wherein the alerting device is configured to provide a wisp of air to the face of the individual if the PPG signals or a physiological parameter derived therefrom are outside the preset value range.

41. The system of claim 23, wherein the PPG sensor and the device for administering the narcotic reversal agent are configured to be worn by the individual.

42. The system of claim 41, further comprising a device configured to administer a CNS depressant, wherein the device configured to administer the CNS depressant is configured to be worn by the individual.

43. The system of claim 41, wherein the controller is configured to be worn by the individual.

44. The system of claim 23, Wherein the controller is configured to be in wireless communication with the PPG sensor.

45. The system of claim 23, wherein the controller is configured to be in wireless communication with the device for administering the narcotic reversal agent.

46. The system of claim 23, wherein the CNS depressant comprises an analgesic agent.
